(19) United States
(12) Patent Application Publication
(10) Pub. No.: US 2026/0328692 A1
(43) Pub. Date: Oct. 22, 2026

(54) DETECTING RADIATION EXPOSURE VIA RAMAN SPECTROSCOPY

(71) Applicant: OHIO STATE INNOVATION FOUNDATION, Columbus, OH (US)

(72) Inventors: Zachary SCHULTZ, Columbus, OH (US); Courtney MORDER, Johnstown, PA (US); Naduparambil Korah JACOB, Columbus, OH (US); Spencer WITTE, Columbus, OH (US)

(21) Appl. No.: 18/994,391

(22) PCT Filed: Jul. 14, 2023
(86) PCT No.: PCT/US2023/027747

§ 371 Date: Jan. 14, 2025

Related U.S. Application Data
(60) Provisional application No. 63/389,543, filed on Jul. 15, 2022.

(51) Int. Cl. G01N 21/65; G01N 33/483

(52) U.S. Cl. CPC G01N 21/65; G01N 33/4833

(57) ABSTRACT

The disclosed subject matter relates to detecting radiation exposure via Raman Spectroscopy. Described herein, for example, are methods comprising: collecting a Raman signal from a sample; and processing the Raman signal to determine a property of the sample; wherein the sample comprises hair, skin, nails, or a combination thereof from a subject; and wherein the property comprises the presence or absence of radiation exposure, the type of radiation, time since the radiation exposure, the duration of the exposure, the dose of the exposure, the dose-rate of exposure, or a combination thereof. Devices configured to perform these methods are also described.

### CROSS-REFERENCE TO RELATED APPLICATIONS

This application claims the benefit of priority to U.S. Provisional Application No. 63/389,543 filed Jul. 15, 2022, which is hereby incorporated herein by reference in its entirety.

### STATEMENT OF GOVERNMENT SUPPORT

This invention was made with government support under W911NF-22-2-0140 awarded by Army Contracting Command. The government has certain rights in the invention.

### BACKGROUND

Radiation biodosimetry determines a past radiation dose from an exposure incident. Radiation biodosimetry is a method for estimating exposure to an individual and the current gold standard method is the dicentric chromosome assay (DCA). However, DCA is not well suited for mass screening, requires a high level of technical input, low throughput, and has a specific time window for use. Point of care methods for biodosimetry include lymphocyte depletion kinetics and clinical examination; however, the time window presents a limiting factor, as does the requirement for an early lymphocyte count to establish a baseline and dose response analysis often requires reading from multiple days.

Radiation response biomarkers and other advanced methods are currently being studied using various methods. There is a critical need to develop rapid, reliable, deployable, and noninvasive diagnostic tools for retrospective detection (qualitative or semi-quantitative) and estimation of absorbed ionizing radiation dose (quantitative).

The devices, methods, and systems discussed herein address these and other needs.

### SUMMARY

In accordance with the purposes of the disclosed devices, methods, and systems as embodied and broadly described herein, the disclosed subject matter relates to detecting radiation exposure via Raman Spectroscopy.

For example, described herein are methods comprising: collecting a Raman signal from a sample; and processing the Raman signal to determine a property of the sample; wherein the sample comprises hair, skin, nails, or a combination thereof from a subject; and wherein the property comprises the presence or absence of radiation exposure, the type of radiation, time since the radiation exposure, the duration of the exposure, the dose of the exposure, the dose-rate of exposure, or a combination thereof.

In some examples, the sample comprises hair, skin, or a combination thereof. In some examples, the sample comprises hair. In some examples, the sample comprises a hair root, a hair shaft, or a combination thereof.

In some examples, the type of radiation comprises ionizing radiation. In some examples, the radiation exposure comprises exposure from neutrons, protons, electrons, photons, alpha-particles, beta-particles, charged particles, gamma rays, X-rays, chemotherapy, UV rays, or a combination thereof.

In some examples, the dose of the exposure is from 0.01 Gy to 20 Gy. In some examples, the time since exposure is from 1 minute to 365 days. In some examples, the dose-rate is from 0.1 to 1000 mGy/min. In some examples, the dose-rate is from 0.1 to 1000 Gy/second.

In some examples, the amount of time from collecting the Raman signal to determining the property of the sample is from 1 second to 1 hour.

In some examples, at least a portion of the Raman signal corresponds to a vibrational frequency associated with radiation damage of the sample.

In some examples, at least a portion of the Raman signal corresponds to at least a portion of a protein that is susceptible to radiation damage. In some examples, at least a portion of the Raman signal comprises a vibrational frequency associated with a disulfide group, a carbonyl group, or a combination thereof.

In some examples, at least a portion of the Raman signal corresponds to at least a portion of a pigment that is susceptible to radiation damage. In some examples, at least a portion of the Raman signal comprises a vibrational frequency associated with a pigment, such as hair pigment.

In some examples, at least a portion of the Raman signal provides evidence of aromatic amino acid modification.

In some examples, processing the Raman signal comprises determining the presence or absence of a signal, the intensity of a signal, the spectral shift of a signal, or a combination thereof. In some examples, processing the Raman signal comprises multivariate analysis of peak characteristics. In some examples, processing the Raman signal to determine the property comprises comparing to a standard curve.

In some examples, the method includes collecting a baseline sample, for example before exposure and/or from an unexposed sample.

In some examples, the method does not include collecting a baseline sample before exposure.

In some examples, processing the Raman signal to determine the property of the sample comprises machine learning.

In some examples, the sample comprises hair and/or nails having a length, and the method further comprises collecting a plurality of Raman signals along the length of the sample, and processing the plurality of Raman signals to determine a property of the sample, wherein the length of the sample corresponds to a growth direction at growth rate, such that the property of the sample comprises time since the radiation exposure. In some examples, the sample comprises hair and the method comprises collecting one or more signals along the shaft of the hair and/or a signal from the follicle of the hair.

In some examples, the method is non-invasive.

In some examples, the method further comprises collecting the sample.

In some examples, the method further comprises purifying and/or treating the sample before collecting the Raman signal.

In some examples, the method further comprises diagnosing and/or monitoring a condition in a subject based on the property of the sample. In some examples, the method further comprises selecting a course of therapy for the subject based on the property of the sample.

Also disclosed herein are devices configured to perform any of the methods described herein. The devices can, for example, comprise: a receptacle configured to at least partially contain the sample; an excitation source; a detector; and a computing device, wherein the computing device is configured to receive and process an electromagnetic signal from the detector; wherein, when the device is assembled together with the sample, then: the receptacle is configured to position the sample such that the sample is in optical communication with the excitation source and the detector; the excitation source is configured to apply an excitation signal to the sample; the detector is configured to collect the Raman signal from the sample; and the computing device is configured to process the Raman to determine the property of the sample. In some examples, the excitation source and/or the detector comprise(s) a Raman spectrometer. In some examples, the device further comprises a polarizer, for example such that the excitation signal is polarized. In some examples, the sample is aligned relative to the polarization of the excitation signal (e.g., parallel, perpendicular, etc.). In some examples, the device is further configured to output the property of the sample and/or a feedback signal based on the property of the sample. In some examples, the amount of time from placing the sample in the receptacle to output is from 1 second to 1 hour. In some examples, the feedback signal comprises haptic feedback, auditory feedback, visual feedback, or a combination thereof.

Additional advantages of the disclosed devices, systems, and methods will be set forth in part in the description which follows, and in part will be obvious from the description. The advantages of the disclosed devices, systems, and methods will be realized and attained by means of the elements and combinations particularly pointed out in the appended claims. It is to be understood that both the foregoing general description and the following detailed description are exemplary and explanatory only and are not restrictive of the disclosed devices, systems, and methods, as claimed.

The details of one or more embodiments of the invention are set forth in the accompanying drawings and the description below. Other features, objects, and advantages of the invention will be apparent from the description and drawings, and from the claims.

### BRIEF DESCRIPTION OF THE FIGURES

The accompanying figures, which are incorporated in and constitute a part of this specification, illustrate several aspects of the disclosure, and together with the description, serve to explain the principles of the disclosure.

FIG. 1A. The Raman spectra of mouse hair follicles from control (black) and animals exposed to 14 Gy localized ionizing radiation (X-rays, using a Small Animal Radiation Research Platform) 7 days prior to collection (red) are shown. The Raman spectra shown were collected on as collected hair samples using <1 mW power on the sample from a 785 nm CW laser. Each spectrum is the average of 3, 60 s acquisitions from 3 different hairs.

FIG. 1B. The Raman spectra for the hair shaft of the control (black) and irradiated animals (red) show significant differences. The Raman spectra shown were collected on as collected hair samples using <1 mW power on the sample from a 785 nm CW laser. Each spectrum is the average of 3, 60 s acquisitions from 3 different hairs.

FIG. 2. Multivariate Curve Resolution (MCR) was performed on the spectra obtained from ionizing radiation exposed and control mice hair. Decomposition of the spectra show two components that account for >95% of the variance in the data. The MCR plot of component 1 versus component 2 indicates the hair from mice exposed to ionizing radiation score lower on component 1, consistent with the expected changes in disulfides and decarboxylation of the protein. The loadings for component 1 and component 2 are shown. The vibrational frequencies readily attributable to disulfides and carbonyl stretches are indicated on the plot.

FIG. 3. A 4×3 mosaic at 50× magnification of a mouse hair is shown along with the Raman spectra acquired at the indicated points along the length of hair. Changes in the Raman spectra can be used to assess time from radiation exposure. Shown here, the spectra acquired from the follicle and along the length of the shaft show differences that correlate with chemical composition and possible changes in composition associated with exposure to ionizing radiation.

FIG. 4. A 4×3 mosaic at 50× magnification of a mouse hair is shown along with the Raman spectra acquired at the indicated points along the length of hair. Changes in Raman spectra can be used to assess time from radiation exposure. Shown here, the spectra acquired from the follicle and along the length of the shaft show differences that correlate with chemical composition and possible changes in composition associated with exposure to ionizing radiation.

FIG. 5. The Raman spectra of mouse hair from follicle (blue) and middle (orange).

FIG. 6. The Raman spectra of mouse hair from follicle (blue) and middle (orange).

FIG. 7. The Raman spectra of mouse hair follicles from control (black) and animals exposed to ionizing radiation 7 days prior to collection (red) are shown.

FIG. 8. 2 Component Multivariate Curve Resolution (MCR) was performed on the spectra obtained from ionizing radiation exposed and control mice hair. Decomposition of the spectra show two components that account for >95% of the variance in the data. The loadings for component 1 and component 2 are shown in FIG. 9A-FIG. 9D.

FIG. 9A. Loadings of component 1 and component 2 from 2 Component Multivariate Curve Resolution (MCR) analysis.

FIG. 9B. Loadings of component 1 and component 2 from 2 Component Multivariate Curve Resolution (MCR) analysis.

FIG. 9C. Loadings of component 1 and component 2 from 2 Component Multivariate Curve Resolution (MCR) analysis.

FIG. 9D. Loadings of component 1 and component 2 from 2 Component Multivariate Curve Resolution (MCR) analysis.

FIG. 10. 2 Component Multivariate Curve Resolution (MCR) analysis indicating data collected from different locations on the hair.

FIG. 11. 3 Component Multivariate Curve Resolution (MCR) was performed on the spectra obtained from ionizing radiation exposed and control mice hair. Loadings for components 1, 2, and 3 are shown in FIG. 14A-FIG. 14D.

FIG. 12. 3 Component Multivariate Curve Resolution (MCR) was performed on the spectra obtained from ionizing radiation exposed and control mice hair. Loadings for components 1, 2, and 3 are shown in FIG. 14A-FIG. 14D.

FIG. 13. 3 Component Multivariate Curve Resolution (MCR) was performed on the spectra obtained from ionizing radiation exposed and control mice hair. Loadings for components 1, 2, and 3 are shown in FIG. 14A-FIG. 14D.

FIG. 14A-FIG. 14D. Loadings of components 1, 2, and 3 from 3 Component Multivariate Curve Resolution (MCR) analysis.

FIG. 15. Schematic illustration of an example computing device.

FIG. 16. Microscopy image of hair sample showing shaft and bulb.

FIG. 17. Microscopy image of hair sample showing hair and end apical tip of hair.

FIG. 18. Microscopy image of hair sample showing hair shaft with light spots and dark spots.

FIG. 19. Schematic diagram of setup.

FIG. 20. Image of software.

FIG. 21. Schematic diagram showing sample stage and orientation.

### DETAILED DESCRIPTION

The devices, methods, and systems described herein may be understood more readily by reference to the following detailed description of specific aspects of the disclosed subject matter and the Examples included therein.

Before the present devices, methods, and systems are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### GENERAL DEFINITIONS

In this specification and in the claims that follow, reference will be made to a number of terms, which shall be defined to have the following meanings.

Throughout the description and claims of this specification the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “an agent” includes mixtures of two or more such agents, reference to “the component” includes mixtures of two or more such components, and the like.

“Optional” or “optionally” means that the subsequently described event or circumstance can or cannot occur, and that the description includes instances where the event or circumstance occurs and instances where it does not.

Ranges can be expressed herein as from “about” one particular value, and/or to “about” another particular value. By “about” is meant within 5% of the value, e.g., within 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

“Exemplary” means “an example of” and is not intended to convey an indication of a preferred or ideal embodiment. “Such as” is not used in a restrictive sense, but for explanatory purposes.

It is understood that throughout this specification the identifiers “first” and “second” are used solely to aid in distinguishing the various components and steps of the disclosed subject matter. The identifiers “first” and “second” are not intended to imply any particular order, amount, preference, or importance to the components or steps modified by these terms.

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician.

The term “inhibit” refers to a decrease in an activity, response, condition, disease, or other biological parameter. This can include but is not limited to the complete ablation of the activity, response, condition, or disease. This can also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed. For example, the terms “prevent” or “suppress” can refer to a treatment that forestalls or slows the onset of a disease or condition or reduced the severity of the disease or condition. Thus, if a treatment can treat a disease in a subject having symptoms of the disease, it can also prevent or suppress that disease in a subject who has yet to suffer some or all of the symptoms.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “artificial intelligence” is defined herein to include any technique that enables one or more computing devices or comping systems (i.e., a machine) to mimic human intelligence. Artificial intelligence (AI) includes, but is not limited to, knowledge bases, machine learning, representation learning, and deep learning.

The term “machine learning” is defined herein to be a subset of AI that enables a machine to acquire knowledge by extracting patterns from raw data. Machine learning techniques include, but are not limited to, logistic regression, support vector machines (SVMs), decision trees, Naïve Bayes classifiers, and artificial neural networks. The term “representation learning” is defined herein to be a subset of machine learning that enables a machine to automatically discover representations needed for feature detection, prediction, or classification from raw data. Representation learning techniques include, but are not limited to, autoencoders. The term “deep learning” is defined herein to be a subset of machine learning that that enables a machine to automatically discover representations needed for feature detection, prediction, classification, etc. using layers of processing. Deep learning techniques include, but are not limited to, artificial neural network or multilayer perceptron (MLP).

Machine learning models include supervised, semi-supervised, and unsupervised learning models. In a supervised learning model, the model learns a function that maps an input (also known as feature or features) to an output (also known as target or targets) during training with a labeled data set (or dataset). In an unsupervised learning model, the model learns patterns (e.g., structure, distribution, etc.) within an unlabeled data set. In a semi-supervised model, the model learns a function that maps an input (also known as feature or features) to an output (also known as target or target) during training with both labeled and unlabeled data.

A logistic regression (LR) classifier is a supervised classification model that uses the logistic function to predict the probability of a target, which can be used for classification. LR classifiers are trained with a data set (also referred to herein as a “dataset”) to maximize or minimize an objective function, for example, a measure of the LR classifier's performance (e.g., error such as L1 or L2 loss), during training. This disclosure contemplates that any algorithm that finds the minimum of the cost function can be used. LR classifiers are known in the art and are therefore not described in further detail herein.

An Naïve Bayes' (NB) classifier is a supervised classification model that is based on Bayes' Theorem, which assumes independence among features (i.e., the presence of one feature in a class is unrelated to the presence of any other features). NB classifiers are trained with a data set by computing the conditional probability distribution of each feature given a label and applying Bayes' Theorem to compute the conditional probability distribution of a label given an observation. NB classifiers are known in the art and are therefore not described in further detail herein.

A k-NN classifier is a supervised classification model that classifies new data points based on similarity measures (e.g., distance functions). k-NN classifier is a non-parametric algorithm, i.e., it does not make strong assumptions about the function mapping input to output and therefore has flexibility to find a function that best fits the data. The k-NN classifiers are trained with a data set (also referred to herein as a “dataset”) by learning associations between all samples and classification labels in the training dataset. This disclosure contemplates that any algorithm that finds the maximum or minimum of the objective function can be used. The k-NN classifiers are known in the art and are therefore not described in further detail herein.

A majority voting ensemble is a meta-classifier that combines a plurality of machine learning classifiers for classification via majority voting. In other words, the majority voting ensemble's final prediction (e.g., class label) is the one predicted most frequently by the member classification models. The majority voting ensembles are known in the art and are therefore not described in further detail herein.

### Chemical Definitions

Unless otherwise defined, all technical and scientific terms used herein have the same meaning as commonly understood by one of ordinary skill in the art to which this invention belongs.

The organic moieties mentioned when defining variable positions within the general formulae described herein (e.g., the term “halogen”) are collective terms for the individual substituents encompassed by the organic moiety. The prefix Cₙ-Cₘ preceding a group or moiety indicates, in each case, the possible number of carbon atoms in the group or moiety that follows.

The term “ion,” as used herein, refers to any molecule, portion of a molecule, cluster of molecules, molecular complex, moiety, or atom that contains a charge (positive, negative, or both at the same time within one molecule, cluster of molecules, molecular complex, or moiety (e.g., zwitterions)) or that can be made to contain a charge. Methods for producing a charge in a molecule, portion of a molecule, cluster of molecules, molecular complex, moiety, or atom are disclosed herein and can be accomplished by methods known in the art, e.g., protonation, deprotonation, oxidation, reduction, alkylation, acetylation, esterification, de-esterification, hydrolysis, etc.

The term “anion” is a type of ion and is included within the meaning of the term “ion.” An “anion” is any molecule, portion of a molecule (e.g., zwitterion), cluster of molecules, molecular complex, moiety, or atom that contains a net negative charge or that can be made to contain a net negative charge. The term “anion precursor” is used herein to specifically refer to a molecule that can be converted to an anion via a chemical reaction (e.g., deprotonation).

The term “cation” is a type of ion and is included within the meaning of the term “ion.” A “cation” is any molecule, portion of a molecule (e.g., zwitterion), cluster of molecules, molecular complex, moiety, or atom, that contains a net positive charge or that can be made to contain a net positive charge. The term “cation precursor” is used herein to specifically refer to a molecule that can be converted to a cation via a chemical reaction (e.g., protonation or alkylation).

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valencies of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

“Z¹,” “Z²,” “Z³,” and “Z⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” as used herein refers to a non-aromatic hydrocarbon group and includes branched and unbranched, alkyl, alkenyl, or alkynyl groups.

As used herein, the term “alkyl” refers to saturated, straight-chained or branched saturated hydrocarbon moieties. Unless otherwise specified, C₁-C₂₄ (e.g., C₁-C₂₂, C₁-C₂₀, C₁-C₁₈, C₁-C₁₆, C₁-C₁₄, C₁-C₁₂, C₁-C₁₀, C₁-C₈, C₁-C₆, or C₁-C₄) alkyl groups are intended. Examples of alkyl groups include methyl, ethyl, propyl, 1-methyl-ethyl, butyl, 1-methyl-propyl, 2-methyl-propyl, 1,1-dimethyl-ethyl, pentyl, 1-methyl-butyl, 2-methyl-butyl, 3-methyl-butyl, 2,2-dimethyl-propyl, 1-ethyl-propyl, hexyl, 1,1-dimethyl-propyl, 1,2-dimethyl-propyl, 1-methyl-pentyl, 2-methyl-pentyl, 3-methyl-pentyl, 4-methyl-pentyl, 1,1-dimethyl-butyl, 1,2-dimethyl-butyl, 1,3-dimethyl-butyl, 2,2-dimethyl-butyl, 2,3-dimethyl-butyl, 3,3-dimethyl-butyl, 1-ethyl-butyl, 2-ethyl-butyl, 1,1,2-trimethyl-propyl, 1,2,2-trimethyl-propyl, 1-ethyl-1-methyl-propyl, 1-ethyl-2-methyl-propyl, heptyl, octyl, nonyl, decyl, dodecyl, tetradecyl, hexadecyl, eicosyl, tetracosyl, and the like. Alkyl substituents may be unsubstituted or substituted with one or more chemical moieties. The alkyl group can be substituted with one or more groups including, but not limited to, hydroxyl, halogen, acetal, acyl, alkyl, alkoxy, alkenyl, alkynyl, aryl, heteroaryl, aldehyde, amino, cyano, carboxylic acid, ester, ether, carbonate ester, carbamate ester, ketone, nitro, phosphonyl, silyl, sulfo-oxo, sulfonyl, sulfone, sulfoxide, or thiol, as described below, provided that the substituents are sterically compatible and the rules of chemical bonding and strain energy are satisfied.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halides (halogens; e.g., fluorine, chlorine, bromine, or iodine). The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

As used herein, the term “alkenyl” refers to unsaturated, straight-chained, or branched hydrocarbon moieties containing a double bond. Unless otherwise specified, C₂-C₂₄ (e.g., C₂-C₂₂, C₂-C₂₀, C₂-C₁₈, C₂-C₁₆, C₂-C₁₄, C₂-C₁₂, C₂-C₁₀, C₂-C₈, C₂-C₆, or C₂-C₄) alkenyl groups are intended. Alkenyl groups may contain more than one unsaturated bond. Examples include ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl. 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl. 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, and 1-ethyl-2-methyl-2-propenyl. The term “vinyl” refers to a group having the structure —CH═CH₂: 1-propenyl refers to a group with the structure —CH═CH—CH₃; and 2-propenyl refers to a group with the structure —CH₂—CH═CH₂. Asymmetric structures such as (Z¹Z²)C═C(Z³Z⁴) are intended to include both the E and Z isomers. This can be presumed in structural formulae herein wherein an asymmetric alkene is present, or it can be explicitly indicated by the bond symbol C═C. Alkenyl substituents may be unsubstituted or substituted with one or more chemical moieties. Examples of suitable substituents include, for example, alkyl, alkoxy, alkenyl, alkynyl, aryl, heteroaryl, acetal, acyl, aldehyde, amino, cyano, carboxylic acid, ester, ether, carbonate ester, carbamate ester, halide, hydroxyl, ketone, nitro, phosphonyl, silyl, sulfo-oxo, sulfonyl, sulfone, sulfoxide, or thiol, as described below, provided that the substituents are sterically compatible and the rules of chemical bonding and strain energy are satisfied.

As used herein, the term “alkynyl” represents straight-chained or branched hydrocarbon moieties containing a triple bond. Unless otherwise specified, C₂-C₂₄ (e.g., C₂-C₂₄, C₂-C₂₀, C₂-C₁₈, C₂-C₁₆, C₂-C₁₄, C₂-C₁₂, C₂-C₁₀, C₂-C₈, C₂-C₆, or C₂-C₄) alkynyl groups are intended. Alkynyl groups may contain more than one unsaturated bond. Examples include C₂-C₆-alkynyl, such as ethynyl, 1-propynyl, 2-propynyl (or propargyl), 1-butynyl, 2-butynyl, 3-butynyl, 1-methyl-2-propynyl, 1-pentynyl, 2-pentynyl, 3-pentynyl, 4-pentynyl, 3-methyl-1-butynyl, 1-methyl-2-butynyl, 1-methyl-3-butynyl, 2-methyl-3-butynyl, 1,1-dimethyl-2-propynyl, 1-ethyl-2-propynyl, 1-hexynyl, 2-hexynyl, 3-hexynyl, 4-hexynyl, 5-hexynyl, 3-methyl-1-pentynyl, 4-methyl-1-pentynyl, 1-methyl-2-pentynyl, 4-methyl-2-pentynyl, 1-methyl-3-pentynyl, 2-methyl-3-pentynyl. 1-methyl-4-pentynyl, 2-methyl-4-pentynyl, 3-methyl-4-pentynyl. 1,1-dimethyl-2-butynyl, 1,1-dimethyl-3-butynyl, 1,2-dimethyl-3-butynyl. 2,2-dimethyl-3-butynyl, 3,3-dimethyl-1-butynyl, 1-ethyl-2-butynyl, 1-ethyl-3-butynyl, 2-ethyl-3-butynyl, and 1-ethyl-1-methyl-2-propynyl. Alkynyl substituents may be unsubstituted or substituted with one or more chemical moieties. Examples of suitable substituents include, for example, alkyl, alkoxy, alkenyl, alkynyl, aryl, heteroaryl, acetal, acyl, aldehyde, amino, cyano, carboxylic acid, ester, ether, carbonate ester, carbamate ester, halide, hydroxyl, ketone, nitro, phosphonyl, silyl, sulfo-oxo, sulfonyl, sulfone, sulfoxide, or thiol, as described below.

As used herein, the term “aryl,” as well as derivative terms such as aryloxy, refers to groups that include a monovalent aromatic carbocyclic group of from 3 to 50 carbon atoms. Aryl groups can include a single ring or multiple condensed rings. In some embodiments, aryl groups include C₆-C₁₀ aryl groups. Examples of aryl groups include, but are not limited to, benzene, phenyl, biphenyl, naphthyl, tetrahydronaphthyl, phenylcyclopropyl, phenoxybenzene, and indanyl. The term “aryl” also includes “heteroaryl,” which is defined as a group that contains an aromatic group that has at least one heteroatom incorporated within the ring of the aromatic group. Examples of heteroatoms include, but are not limited to, nitrogen, oxygen, sulfur, and phosphorus. The term “non-heteroaryl,” which is also included in the term “aryl,” defines a group that contains an aromatic group that does not contain a heteroatom. The aryl substituents may be unsubstituted or substituted with one or more chemical moieties. Examples of suitable substituents include, for example, alkyl, alkoxy, alkenyl, alkynyl, aryl, heteroaryl, acetal, acyl, aldehyde, amino, cyano, carboxylic acid, ester, ether, carbonate ester, carbamate ester, halide, hydroxyl, ketone, nitro, phosphonyl, silyl, sulfo-oxo, sulfonyl, sulfone, sulfoxide, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, acetal, acyl, aldehyde, amino, cyano, carboxylic acid, ester, ether, carbonate ester, carbamate ester, halide, hydroxyl, ketone, nitro, phosphonyl, silyl, sulfo-oxo, sulfonyl, sulfone, sulfoxide, or thiol as described herein.

The term “cycloalkenyl” as used herein is a non-aromatic carbon-based ring composed of at least three carbon atoms and containing at least one double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above and is included within the meaning of the term “cycloalkenyl,” where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, acetal, acyl, aldehyde, amino, cyano, carboxylic acid, ester, ether, carbonate ester, carbamate ester, halide, hydroxyl, ketone, nitro, phosphonyl, silyl, sulfo-oxo, sulfonyl, sulfone, sulfoxide, or thiol as described herein.

The term “cyclic group” is used herein to refer to either aryl groups, non-aryl groups (i.e., cycloalkyl, heterocycloalkyl, cycloalkenyl, and heterocycloalkenyl groups), or both. Cyclic groups have one or more ring systems (e.g., monocyclic, bicyclic, tricyclic, polycyclic, etc.) that can be substituted or unsubstituted. A cyclic group can contain one or more aryl groups, one or more non-aryl groups, or one or more aryl groups and one or more non-aryl groups.

The term “acyl” as used herein is represented by the formula —C(O)Z¹ where Z¹ can be a hydrogen, hydroxyl, alkoxy, alkyl, alkenyl, alkynyl, aryl, heteroaryl, cycloalkyl, cycloalkenyl, heterocycloalkyl, or heterocycloalkenyl group described above. As used herein, the term “acyl” can be used interchangeably with “carbonyl.” Throughout this specification “C(O)” or “CO” is a shorthand notation for C═O.

The term “acetal” as used herein is represented by the formula (Z¹Z²)C(═OZ³)(═OZ⁴), where Z¹, Z², Z³, and Z⁴ can be, independently, a hydrogen, halogen, hydroxyl, alkyl, alkenyl, alkynyl, aryl, heteroaryl, cycloalkyl, cycloalkenyl, heterocycloalkyl, or heterocycloalkenyl group described above.

The term “alkanol” as used herein is represented by the formula Z¹OH, where Z¹ can be an alkyl, alkenyl, alkynyl, aryl, heteroaryl, cycloalkyl, cycloalkenyl, heterocycloalkyl, or heterocycloalkenyl group described above.

As used herein, the term “alkoxy” as used herein is an alkyl group bound through a single, terminal ether linkage; that is, an “alkoxy” group can be defined as to a group of the formula Z¹—O—, where Z¹ is unsubstituted or substituted alkyl as defined above. Unless otherwise specified, alkoxy groups wherein Z¹ is a C₁-C₂₄ (e.g., C₁-C₂₂, C₁-C₂₀, C₁-C₁₈, C₁-C₁₆, C₁-C₁₄, C₁-C₁₂, C₁-C₁₀, C₁-C₈, C₁-C₆, or C₁-C₄) alkyl group are intended. Examples include methoxy, ethoxy, propoxy, 1-methyl-ethoxy, butoxy, 1-methyl-propoxy, 2-methyl-propoxy, 1,1-dimethyl-ethoxy, pentoxy, 1-methyl-butyloxy. 2-methyl-butoxy, 3-methyl-butoxy, 2,2-di-methyl-propoxy, 1-ethyl-propoxy, hexoxy, 1,1-dimethyl-propoxy, 1,2-dimethyl-propoxy, 1-methyl-pentoxy, 2-methyl-pentoxy, 3-methyl-pentoxy, 4-methyl-pentoxy, 1,1-dimethyl-butoxy, 1,2-dimethyl-butoxy, 1,3-dimethyl-butoxy, 2,2-dimethyl-butoxy, 2,3-dimethyl-butoxy, 3,3-dimethyl-butoxy, 1-ethyl-butoxy, 2-ethylbutoxy, 1,1,2-trimethyl-propoxy, 1,2,2-trimethyl-propoxy, 1-ethyl-1-methyl-propoxy, and 1-ethyl-2-methyl-propoxy.

The term “aldehyde” as used herein is represented by the formula —C(O)H. Throughout this specification “C(O)” is a shorthand notation for C═O.

The terms “amine” or “amino” as used herein are represented by the formula —NZ¹Z²Z³, where Z¹, Z², and Z³ can each be substitution group as described herein, such as hydrogen, an alkyl, alkenyl, alkynyl, aryl, heteroaryl, cycloalkyl, cycloalkenyl, heterocycloalkyl, or heterocycloalkenyl group described above.

The terms “amide” or “amido” as used herein are represented by the formula —C(O)NZ¹Z², where Z¹ and Z² can each be substitution group as described herein, such as hydrogen, an alkyl, alkenyl, alkynyl, aryl, heteroaryl, cycloalkyl, cycloalkenyl, heterocycloalkyl, or heterocycloalkenyl group described above.

The term “anhydride” as used herein is represented by the formula Z¹C(O)OC(O)Z² where Z¹ and Z², independently, can be an alkyl, alkenyl, alkynyl, aryl, heteroaryl, cycloalkyl, cycloalkenyl, heterocycloalkyl, or heterocycloalkenyl group described above.

The term “cyclic anhydride” as used herein is represented by the formula:

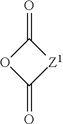

where Z¹ can be an alkyl, alkenyl, alkynyl, aryl, heteroaryl, cycloalkyl, cycloalkenyl, heterocycloalkyl, or heterocycloalkenyl group described above.

The term “azide” as used herein is represented by the formula —N═N═N.

The term “carboxylic acid” as used herein is represented by the formula —C(O)OH.

A “carboxylate” or “carboxyl” group as used herein is represented by the formula —C(O)O⁻.

A “carbonate ester” group as used herein is represented by the formula Z¹OC(O)OZ².

The term “cyano” as used herein is represented by the formula —CN.

The term “ester” as used herein is represented by the formula —OC(O)Z¹ or —C(O)OZ¹, where Z¹ can be an alkyl, alkenyl, alkynyl, aryl, heteroaryl, cycloalkyl, cycloalkenyl, heterocycloalkyl, or heterocycloalkenyl group described above.

The term “ether” as used herein is represented by the formula Z¹OZ², where Z¹ and Z² can be, independently, an alkyl, alkenyl, alkynyl, aryl, heteroaryl, cycloalkyl, cycloalkenyl, heterocycloalkyl, or heterocycloalkenyl group described above.

The term “epoxy” or “epoxide” as used herein refers to a cyclic ether with a three atom ring and can represented by the formula:

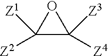

where Z¹, Z², Z³, and Z⁴ can be, independently, an alkyl, alkenyl, alkynyl, aryl, heteroaryl, cycloalkyl, cycloalkenyl, heterocycloalkyl, or heterocycloalkenyl group described above

The term “ketone” as used herein is represented by the formula Z¹C(O)Z², where Z¹ and Z² can be, independently, an alkyl, alkenyl, alkynyl, aryl, heteroaryl, cycloalkyl, cycloalkenyl, heterocycloalkyl, or heterocycloalkenyl group described above.

The term “halide” or “halogen” or “halo” as used herein refers to fluorine, chlorine, bromine, and iodine.

The term “hydroxyl” as used herein is represented by the formula —OH.

The term “nitro” as used herein is represented by the formula —NO₂.

The term “phosphonyl” is used herein to refer to the phospho-oxo group represented by the formula —P(O)(OZ¹)₂, where Z¹ can be hydrogen, an alkyl, alkenyl, alkynyl, aryl, heteroaryl, cycloalkyl, cycloalkenyl, heterocycloalkyl, or heterocycloalkenyl group described above.

The term “silyl” as used herein is represented by the formula —SiZ¹Z²Z³, where Z¹, Z², and Z³ can be, independently, hydrogen, alkyl, alkoxy, alkenyl, alkynyl, aryl, heteroaryl, cycloalkyl, cycloalkenyl, heterocycloalkyl, or heterocycloalkenyl group described above.

The term “sulfonyl” or “sulfone” is used herein to refer to the sulfo-oxo group represented by the formula —S(O)₂Z¹, where Z¹ can be hydrogen, an alkyl, alkenyl, alkynyl, aryl, heteroaryl, cycloalkyl, cycloalkenyl, heterocycloalkyl, or heterocycloalkenyl group described above.

The term “sulfide” as used herein is comprises the formula —S—.

The term “thiol” as used herein is represented by the formula —SH.

“R¹,” “R²,” “R³,” “Rⁿ,” etc., where n is some integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an amine group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

Unless stated to the contrary, a formula with chemical bonds shown only as solid lines and not as wedges or dashed lines contemplates each possible stereoisomer or mixture of stereoisomer (e.g., each enantiomer, each diastereomer, each meso compound, a racemic mixture, or scalemic mixture).

### Methods

Disclosed herein are methods comprising: collecting a Raman signal from a sample; and processing the Raman signal to determine a property of the sample; wherein the sample comprises hair, skin, nails, or a combination thereof from a subject; and wherein the property comprises the presence or absence of radiation exposure, the type of radiation, time since the radiation exposure, the duration of the exposure, the dose of the exposure, the dose-rate of exposure, or a combination thereof.

In some examples, the type of radiation comprises ionizing radiation. In some examples, the radiation exposure comprises exposure from neutrons, protons, electrons, photons, alpha-particles, beta-particles, charged particles, gamma rays, X-rays, chemotherapy, UV rays, or a combination thereof.

In some examples, the dose of the exposure is 0.01 Gy or more (e.g., 0.02 Gy or more, 0.03 Gy or more, 0.04 Gy or more, 0.05 Gy or more, 0.06 Gy or more, 0.07 Gy or more, 0.08 Gy or more, 0.09 Gy or more, 0.1 Gy or more, 0.15 Gy or more, 0.2 Gy or more, 0.25 Gy or more, 0.3 Gy or more, 0.35 Gy or more, 0.4 Gy or more, 0.45 Gy or more, 0.5 Gy or more, 0.6 Gy or more, 0.7 Gy or more, 0.8 Gy or more, 0.9 Gy or more, 1 Gy or more, 1.25 Gy or more, 1.5 Gy or more, 1.75 Gy or more, 2 Gy or more, 2.25 Gy or more, 2.5 Gy or more, 2.75 Gy or more, 3 Gy or more, 3.25 Gy or more, 3.5 Gy or more, 3.75 Gy or more, 4 Gy or more, 4.25 Gy or more, 4.5 Gy or more, 4.75 Gy or more, 5 Gy or more, 5.5 Gy or more, 6 Gy or more, 6.5 Gy or more, 7 Gy or more, 7.5 Gy or more, 8 Gy or more, 8.5 Gy or more, 9 Gy or more, 9.5 Gy or more, 10 Gy or more, 11 Gy or more, 12 Gy or more, 13 Gy or more, 14 Gy or more, 15 Gy or more, 16 Gy or more, 17 Gy or more, 18 Gy or more, or 19 Gy or more). In some examples, the dose of the exposure is 20 Gy or less (e.g., 19 Gy or less. 18 Gy or less, 17 Gy or less. 16 Gy or less, 15 Gy or less, 14 Gy or less, 13 Gy or less, 12 Gy or less, 11 Gy or less, 10 Gy or less, 9.5 Gy or less, 9 Gy or less, 8.5 Gy or less, 8 Gy or less, 7.5 Gy or less, 7 Gy or less, 6.5 Gy or less, 6 Gy or less, 5.5 Gy or less, 5 Gy or less, 4.75 Gy or less, 4.5 Gy or less, 4.25 Gy or less, 4 Gy or less. 3.75 Gy or less, 3.5 Gy or less, 3.25 Gy or less, 3 Gy or less, 2.75 Gy or less, 2.5 Gy or less, 2.25 Gy or less, 2 Gy or less, 1.75 Gy or less, 1.5 Gy or less, 1.25 Gy or less, 1 Gy or less, 0.9 Gy or less, 0.8 Gy or less, 0.7 Gy or less, 0.6 Gy or less, 0.5 Gy or less, 0.45 Gy or less, 0.4 Gy or less, 0.35 Gy or less, 0.3 Gy or less. 0.25 Gy or less, 0.2 Gy or less, 0.15 Gy or less, 0.1 Gy or less, 0.09 Gy or less, 0.08 Gy or less, 0.07 Gy or less, 0.06 Gy or less, 0.05 Gy or less, 0.04 Gy or less, 0.03 Gy or less, or 0.02 Gy or less). The dose of the exposure can range from any of the minimum values described above to any of the maximum values described above. For example, the dose of the exposure can be from 0.01 Gy to 20 Gy (e.g., from 0.01 Gy to 10 Gy, from 10 Gy to 14 Gy, from 0.01 Gy to 0.1 Gy, from 0.1 Gy to 1 Gy, from 1 Gy to 20 Gy, from 0.01 Gy to 15 Gy, from 0.01 Gy to 10 Gy, from 0.01 Gy to 5 Gy, from 0.01 Gy to 1 Gy, from 0.01 Gy to 0.5 Gy, from 0.05 Gy to 20 Gy, from 0.1 Gy to 20 Gy, from 0.5 Gy to 20 Gy, from 1 Gy to 20 Gy, from 5 Gy to 20 Gy, from 0.05 Gy to 14 Gy, from 0.1 Gy to 15 Gy).

In some examples, the time since exposure is 1 minute or more (e.g., 2 minutes or more, 3 minutes or more, 4 minutes or more, 5 minutes or more, 10 minutes or more, 15 minutes or more, 20 minutes or more, 25 minutes or more, 30 minutes or more, 35 minutes or more, 40 minutes or more, 45 minutes or more, 50 minutes or more, 55 minutes or more, 1 hour or more, 1.25 hours or more, 1.5 hours or more, 1.75 hours or more, 2 hours or more, 2.25 hours or more, 2.5 hours or more, 2.75 hours or more, 3 hours or more, 3.25 hours or more, 3.5 hours or more, 3.75 hours or more, 4 hours or more, 4.25 hours or more, 4.5 hours or more, 4.75 hours or more, 5 hours or more, 5.5 hours or more, 6 hours or more, 6.5 hours or more, 7 hours or more, 7.5 hours or more, 8 hours or more, 8.5 hours or more, 9 hours or more, 9.5 hours or more, 10 hours or more, 11 hours or more, 12 hours or more, 13 hours or more, 14 hours or more, 16 hours or more, 18 hours or more, 20 hours or more, 22 hours or more, 1 day or more, 1.25 days or more, 1.5 days or more, 1.75 days or more, 2 days or more, 2.25 days or more, 2.5 days or more, 2.75 days or more, 3 days or more, 3.25 days or more, 3.5 days or more, 3.75 days or more, 4 days or more, 4.25 days or more, 4.5 days or more, 4.75 days or more, 5 days or more, 5.5 days or more, 6 days or more, 6.5 days or more, 7 days or more, 7.5 days or more, 8 days or more, 8.5 days or more, 9 days or more, 9.5 days or more, 10 days or more, 11 days or more, 12 days or more, 13 days or more, 14 days or more, 15 days or more, 16 days or more, 17 days or more, 18 days or more, 19 days or more, 20 days or more, 25 days or more, 30 days or more, 35 days or more, 40 days or more, 45 days or more, 50 days or more, 60 days or more, 70 days or more, 80 days or more, 90 days or more, 100 days or more, 110 days or more, 120 days or more, 130 days or more, 140 days or more, 150 days or more, 175 days or more, 200 days or more, 225 days or more, 250 days or more, 275 days or more, 300 days or more, 325 days or more, or 350 days or more). In some examples, the time since exposure is 1 year or less (e.g., 350 days or less, 325 days or less, 300 days or less, 275 days or less, 250 days or less, 225 days or less, 200 days or less, 175 days or less, 150 days or less, 140 days or less, 130 days or less. 120 days or less. 110 days or less, 100 days or less, 90 days or less, 80 days or less. 70 days or less, 60 days or less, 50 days or less, 45 days or less, 40 days or less, 35 days or less, 30 days or less, 25 days or less, 20 days or less, 19 days or less, 18 days or less, 17 days or less, 16 days or less, 15 days or less, 14 days or less, 13 days or less, 12 days or less, 11 days or less, 10 days or less, 9.5 days or less, 9 days or less, 8.5 days or less. 8 days or less, 7.5 days or less, 7 days or less. 6.5 days or less, 6 days or less, 5.5 days or less, 5 days or less, 4.75 days or less, 4.5 days or less, 4.25 days or less, 4 days or less, 3.75 days or less, 3.5 days or less, 3.25 days or less, 3 days or less, 2.75 days or less, 2.5 days or less, 2.25 days or less, 2 days or less, 1.75 days or less, 1.5 days or less, 1.25 days or less, 1 day or less, 22 hours or less, 20 hours or less, 18 hours or less, 16 hours or less, 14 hours or less, 13 hours or less, 12 hours or less, 11 hours or less, 10 hours or less, 9.5 hours or less, 9 hours or less, 8.5 hours or less, 8 hours or less, 7.5 hours or less, 7 hours or less, 6.5 hours or less, 6 hours or less, 5.5 hours or less, 5 hours or less, 4.75 hours or less, 4.5 hours or less, 4.25 hours or less, 4 hours or less, 3.75 hours or less, 3.5 hours or less, 3.25 hours or less, 3 hours or less. 2.75 hours or less, 2.5 hours or less, 2.25 hours or less, 2 hours or less, 1.75 hours or less, 1.5 hours or less, 1.25 hours or less, 1 hour or less, 55 minutes or less, 50 minutes or less, 45 minutes or less, 40 minutes or less, 35 minutes or less, 30 minutes or less, 25 minutes or less, 20 minutes or less, 15 minutes or less, 10 minutes or less, 5 minutes or less, 4 minutes or less, 3 minutes or less, or 2 minutes or less). The time since exposure can range from any of the minimum values described above to any of the maximum values described above. For example, the time since exposure can be from 1 minute to 365 days (e.g., from 1 minute to 1 week, from 1 week to 1 year, from 1 minute to 1 hour, from 1 hour to 1 day, from 1 day to 1 week, from 1 week to 1 month, from 1 month to 1 year, 1 minute to 11 months, from 1 minute to 6 months, from 1 minute to 3 months, from 1 minute to 1 month, from 1 minute to 2 weeks, from 1 minute to 1 week, from 1 minute to 3.5 days, from 1 minute to 1 day, from 1 minute to 20 hours, from 1 minute to 18 hours, from 1 minute to 12 hours, from 1 minute to 6 hours, from 1 minute to 1 hour, from 1 minute to 30 minutes, from 5 minutes to 1 year, from 10 minutes to 1 year, from 15 minutes to 1 year, from 30 minutes to 1 year, from 1 hour to 1 year, from 6 hours to 1 year, from 12 hours to 1 year, from 18 hours to 1 year, from 20 hours to 1 year, from 1 day to 1 year, from 3.5 days to 1 year, from 1 week to 1 year, from 2 weeks to 1 year, from 1 month to 1 year, from 3 months to 1 year, from 6 months to 1 year, from 5 minutes to 325 days, or from 10 minutes to 120 days).

In some examples, the dose-rate is 0.1 mGy/minute or more (e.g., 0.2 mGy/min or more, 0.3 mGy/min or more, 0.4 mGy/min or more, 0.5 mGy/min or more, 0.75 mGy/min or more, 1 mGy/min or more, 1.25 mGy/min or more, 1.5 mGy/min or more, 1.75 mGy/min or more, 2 mGy/min or more, 2.5 mGy/min or more, 3 mGy/min or more, 3.5 mGy/min or more, 4 mGy/min or more, 4.5 mGy/min or more, 5 mGy/min or more, 6 mGy/min or more, 7 mGy/min or more, 8 mGy/min or more, 9 mGy/min or more, 10 mGy/min or more, 15 mGy/min or more, 20 mGy/min or more, 25 mGy/min or more, 30 mGy/min or more, 35 mGy/min or more, 40 mGy/min or more, 45 mGy/min or more, 50 mGy/min or more, 60 mGy/min or more, 70 mGy/min or more, 80 mGy/min or more, 90 mGy/min or more, 100 mGy/min or more, 125 mGy/min or more, 150 mGy/min or more, 175 mGy/min or more, 200 mGy/min or more, 225 mGy/min or more, 250 mGy/min or more, 300 mGy/min or more, 350 mGy/min or more, 400 mGy/min or more, 450 mGy/min or more, 500 mGy/min or more, 550 mGy/min or more, 600 mGy/min or more, 650 mGy/min or more, 700 mGy/min or more, 750 mGy/min or more, 800 mGy/min or more, 850 mGy/min or more, 900 mGy/min or more, or 950 mGy/min or more). In some examples, the dose-rate is 1000 mGy/min or less (e.g., 950 mGy/min or less, 900 mGy/min or less, 850 mGy/min or less, 800 mGy/min or less, 750 mGy/min or less, 700 mGy/min or less, 650 mGy/min or less, 600 mGy/min or less, 550 mGy/min or less. 500 mGy/min or less, 450 mGy/min or less, 400 mGy/min or less, 350 mGy/min or less, 300 mGy/min or less, 250 mGy/min or less, 225 mGy/min or less, 200 mGy/min or less, 175 mGy/min or less, 150 mGy/min or less, 125 mGy/min or less, 100 mGy/min or less, 90 mGy/min or less, 80 mGy/min or less, 70 mGy/min or less, 60 mGy/min or less, 50 mGy/min or less, 45 mGy/min or less, 40 mGy/min or less. 35 mGy/min or less, 30 mGy/min or less, 25 mGy/min or less, 20 mGy/min or less, 15 mGy/min or less, 10 mGy/min or less, 9 mGy/min or less, 8 mGy/min or less, 7 mGy/min or less, 6 mGy/min or less, 5 mGy/min or less, 4.5 mGy/min or less, 4 mGy/min or less, 3.5 mGy/min or less, 3 mGy/min or less, 2.5 mGy/min or less, 2 mGy/min or less, 1.75 mGy/min or less, 1.5 mGy/min or less, 1.25 mGy/min or less. 1 mGy/min or less, 0.75 mGy/min or less, 0.5 mGy/min or less. 0.4 mGy/min or less, 0.3 mGy/min or less, or 0.2 mGy/min or less). The dose-rate can range from any of the minimum values described above to any of the maximum values described above. For example, the dose rate can be from 0.1 to 1000 mGy/min (e.g., from 0.1 to 10 mGy/min, from 10 to 1000 mGy/min, from 0.1 to 1 mGy/min, from 1 to 10 mGy/min, from 10 to 100 mGy/min. from 100 to 1000 mGy/min, from 0.1 to 900 mGy/min, from 0.1 to 750 mGy/min, from 0.1 to 500 mGy/min, from 0.1 to 250 mGy/min, from 0.1 to 100 mGy/min, from 0.1 to 75 mGy/min, from 0.1 to 50 mGy/min, from 0.1 to 25 mGy/min, from 0.1 to 10 mGy/min, from 0.1 to 5 mGy/min, from 0.5 to 1000 mGy/min, from 1 to 1000 mGy/min, from 5 to 1000 mGy/min, from 10 to 1000 mGy/min, from 25 to 1000 mGy/min, from 50 to 1000 mGy/min, from 75 to 1000 mGy/min, from 100 to 1000 mGy/min, from 250 to 1000 mGy/min, from 500 to 1000 mGy/min, from 0.5 to 900 mGy/min, or from 1 to 750 mGy/min).

In some examples, the dose-rate is 0.1 Gy/second or more (e.g., 0.2 Gy/sec or more, 0.3 Gy/sec or more, 0.4 Gy/sec or more, 0.5 Gy/sec or more, 0.75 Gy/sec or more, 1 Gy/sec or more, 1.25 Gy/sec or more, 1.5 Gy/sec or more, 1.75 Gy/sec or more, 2 Gy/sec or more, 2.5 Gy/sec or more, 3 Gy/sec or more, 3.5 Gy/sec or more, 4 Gy/sec or more, 4.5 Gy/sec or more, 5 Gy/sec or more, 6 Gy/sec or more, 7 Gy/sec or more, 8 Gy/sec or more, 9 Gy/sec or more, 10 Gy/sec or more, 15 Gy/sec or more, 20 Gy/sec or more, 25 Gy/sec or more, 30 Gy/sec or more, 35 Gy/sec or more, 40 Gy/sec or more, 45 Gy/sec or more, 50 Gy/sec or more, 60 Gy/sec or more, 70 Gy/sec or more, 80 Gy/sec or more, 90 Gy/sec or more, 100 Gy/sec or more, 125 Gy/sec or more, 150 Gy/sec or more, 175 Gy/sec or more, 200 Gy/sec or more, 225 Gy/sec or more, 250 Gy/sec or more, 300 Gy/sec or more, 350 Gy/sec or more, 400 Gy/sec or more, 450 Gy/sec or more, 500 Gy/sec or more, 550 Gy/sec or more, 600 Gy/sec or more, 650 Gy/sec or more, 700 Gy/sec or more, 750 Gy/sec or more, 800 Gy/sec or more, 850 Gy/sec or more, 900 Gy/sec or more, or 950 Gy/sec or more). In some examples, the dose-rate is 1000 Gy/sec or less (e.g., 950 Gy/sec or less, 900 Gy/sec or less, 850 Gy/sec or less, 800 Gy/sec or less, 750 Gy/sec or less, 700 Gy/sec or less, 650 Gy/sec or less, 600 Gy/sec or less, 550 Gy/sec or less, 500 Gy/sec or less, 450 Gy/sec or less, 400 Gy/sec or less, 350 Gy/sec or less, 300 Gy/sec or less, 250 Gy/sec or less, 225 Gy/sec or less, 200 Gy/sec or less, 175 Gy/sec or less, 150 Gy/sec or less, 125 Gy/sec or less, 100 Gy/sec or less, 90 Gy/sec or less, 80 Gy/sec or less, 70 Gy/sec or less, 60 Gy/sec or less, 50 Gy/sec or less, 45 Gy/sec or less, 40 Gy/sec or less, 35 Gy/sec or less, 30 Gy/sec or less, 25 Gy/sec or less, 20 Gy/sec or less, 15 Gy/sec or less, 10 Gy/sec or less, 9 Gy/sec or less, 8 Gy/sec or less, 7 Gy/sec or less, 6 Gy/sec or less, 5 Gy/sec or less, 4.5 Gy/sec or less, 4 Gy/sec or less, 3.5 Gy/sec or less, 3 Gy/sec or less, 2.5 Gy/sec or less, 2 Gy/sec or less, 1.75 Gy/sec or less, 1.5 Gy/sec or less, 1.25 Gy/sec or less, 1 Gy/sec or less, 0.75 Gy/sec or less, 0.5 Gy/sec or less, 0.4 Gy/sec or less, 0.3 Gy/sec or less, or 0.2 Gy/sec or less). The dose-rate can range from any of the minimum values described above to any of the maximum values described above. For example, the dose rate can be from 0.1 to 1000 Gy/sec (e.g., from 0.1 to 10 Gy/sec. from 10 to 1000 Gy/sec. from 0.1 to 1 Gy/sec, from 1 to 10 Gy/sec, from 10 to 100 Gy/sec, from 100 to 1000 Gy/sec, from 0.1 to 900 Gy/sec, from 0.1 to 750 Gy/sec, from 0.1 to 500 Gy/sec, from 0.1 to 250 Gy/sec, from 0.1 to 100 Gy/sec, from 0.1 to 75 Gy/sec, from 0.1 to 50 Gy/sec, from 0.1 to 25 Gy/sec, from 0.1 to 10 Gy/sec, from 0.1 to 5 Gy/sec, from 0.5 to 1000 Gy/sec. from 1 to 1000 Gy/sec, from 5 to 1000 Gy/sec, from 10 to 1000 Gy/sec, from 25 to 1000 Gy/sec. from 50 to 1000 Gy/sec. from 75 to 1000 Gy/sec. from 100 to 1000 Gy/sec, from 250 to 1000 Gy/sec, from 500 to 1000 Gy/sec, from 0.5 to 900 Gy/sec, or from 1 to 750 Gy/sec).

In some examples, the amount of time from collecting the Raman signal to determining the property of the sample is 1 second or more (e.g., 2 seconds or more, 3 seconds or more, 4 seconds or more, 5 seconds or more, 10 seconds or more, 15 seconds or more, 20 seconds or more, 25 seconds or more, 30 seconds or more, 35 seconds or more, 40 seconds or more, 45 seconds or more, 50 seconds or more, 55 seconds or more, 1 minute or more, 1.25 minutes or more, 1.5 minutes or more, 1.75 minutes or more, 2 minutes or more, 2.25 minutes or more, 2.5 minutes or more, 3 minutes or more, 3.5 minutes or more, 4 minutes or more, 4.5 minutes or more, 5 minutes or more, 5.5 minutes or more, 6 minutes or more, 7 minutes or more, 8 minutes or more, 9 minutes or more, 10 minutes or more, 15 minutes or more, 20 minutes or more, 25 minutes or more, 30 minutes or more, 35 minutes or more, 40 minutes or more, 45 minutes or more, 50 minutes or more, or 55 minutes or more). In some examples, the amount of time from collecting the Raman signal to determining the property of the sample is 1 hour or less (e.g., 55 minutes or less, 50 minutes or less, 45 minutes or less, 40 minutes or less, 35 minutes or less, 30 minutes or less, 25 minutes or less, 20 minutes or less, 15 minutes or less, 10 minutes or less, 9 minutes or less, 8 minutes or less, 7 minutes or less, 6 minutes or less, 5.5 minutes or less, 5 minutes or less, 4.5 minutes or less, 4 minutes or less, 3.5 minutes or less, 3 minutes or less, 2.5 minutes or less, 2.25 minutes or less, 2 minutes or less, 1.75 minutes or less, 1.5 minutes or less, 1.25 minutes or less, 1 minute or less, 55 seconds or less, 50 seconds or less, 45 seconds or less, 40 seconds or less. 35 seconds or less. 30 seconds or less. 25 seconds or less. 20 seconds or less. 15 seconds or less, 10 seconds or less, 5 seconds or less, 4 seconds or less, 3 seconds or less, or 2 seconds or less). The amount of time from collecting the Raman signal to determining the property of the sample can range from any of the minimum values described above to any of the maximum values described above. For example, the amount of time from collecting the Raman signal to determining the property of the sample is from 1 second to 1 hour (e.g., from 1 second to 30 minutes, from 30 minutes to 1 hour, from 1 second to 1 minute, from 1 minute to 10 minutes, from 10 minutes to 1 hour, from 1 second to 55 minutes, from 1 second to 45 minutes, from 1 second to 30 minutes, from 1 second to 15 minutes, from 1 second to 10 minutes, from 1 second to 5 minutes from 1 second to 1 minute, from 5 seconds to 1 hour, from 10 seconds to 1 hour, from 15 seconds to 1 hour, from 30 seconds to 1 hour, from 45 seconds to 1 hour, from 1 minute to 1 hour, from 5 minutes to 1 hour, from 10 minutes to 1 hour, from 15 minutes to 1 hour, from 30 minutes to 1 hour, from 5 seconds to 55 minutes, or from 10 seconds to 45 minutes).

In some examples, at least a portion of the Raman signal corresponds to a vibrational frequency associated with radiation damage of the sample.

In some examples, at least a portion of the Raman signal corresponds to at least a portion of a protein that is susceptible to radiation damage. For example, at least a portion of the Raman signal can comprise a vibrational frequency associated with a disulfide group, a carbonyl group, or a combination thereof.

In some examples, at least a portion of the Raman signal corresponds to at least a portion of a pigment that is susceptible to radiation damage. For example, at least a portion of the Raman signal can comprise a vibrational frequency associated with a pigment, such as hair pigment.

In some examples, at least a portion of the Raman signal can provide evidence of aromatic amino acid modification.

In some examples, processing the Raman signal comprises determining the presence or absence of a signal, the intensity of a signal, the spectral shift of a signal, or a combination thereof. In some examples, processing the Raman signal comprises multivariate analysis of peak characteristics. In some examples, processing the Raman signal to determine the property comprises comparing to a standard curve. In some examples, processing the Raman signal comprises machine learning.

In some examples, the methods include collecting a baseline sample. The baseline sample can, for example, be collected before exposure and/or from an unexposed sample.

In some examples, the methods do not include collecting a baseline sample before exposure.

In some examples, the sample comprises hair, skin, or a combination thereof. In some examples, the sample comprises hair, such as a hair root, a hair shaft, or a combination thereof.

In some examples, the sample comprises hair and/or nails having a length, and the method further comprises collecting a plurality of Raman signals along the length of the sample, and processing the plurality of Raman signals to determine a property of the sample, wherein the length of the sample corresponds to a growth direction at growth rate, such that the property of the sample comprises time since the radiation exposure. In some examples, the sample comprises hair and the method comprises collecting one or more signals along the shaft of the hair and/or a signal from the follicle of the hair.

In some examples, the methods are non-invasive.

In some examples, the methods further comprise collecting the sample.

In some examples, the methods further comprise purifying and/or treating the sample before collecting the Raman signal.

In some examples, the methods further comprise diagnosing and/or monitoring a condition in a subject based on the property of the sample. In some examples, the methods further comprise selecting a course of therapy for the subject based on the property of the sample.

### Devices

Also disclosed herein are devices, for example for performing any of the methods described herein.

For example, also disclosed herein are devices comprising: a receptacle configured to at least partially contain the sample; an excitation source; a detector; and a computing device, wherein the computing device is configured to receive and process an electromagnetic signal from the detector; wherein, when the device is assembled together with the sample, then: the receptacle is configured to position the sample such that the sample is in optical communication with the excitation source and the detector: the excitation source is configured to apply an excitation signal to the sample; the detector is configured to collect the Raman signal from the sample; and the computing device is configured to process the Raman to determine the property of the sample. In some examples, the excitation source and/or the detector can comprise(s) a Raman spectrometer. In some examples, the device further comprises a polarizer, for example such that the excitation signal is polarized. The excitation can, for example, be polarized in a specific direction. In some examples, the polarization of the excitation can be controlled and/or changed. In some examples, the sample is aligned relative to the polarization of the excitation signal (e.g., parallel, perpendicular, etc.).

In some examples, the device is further configured to output the property of the sample and/or a feedback signal based on the property of the sample. In some examples, the device can further comprise one or more output devices (e.g., a display, speakers, printer. LED, etc.) configured to output the property of the sample and/or a feedback signal based on the property of the sample. The feedback signal can, for example, comprise haptic feedback, auditory feedback, visual feedback, or a combination thereof.

In some examples, the amount of time from placing the sample in the receptacle to output is 1 second or more (e.g., 2 seconds or more, 3 seconds or more, 4 seconds or more, 5 seconds or more, 10 seconds or more, 15 seconds or more, 20 seconds or more, 25 seconds or more, 30 seconds or more, 35 seconds or more, 40 seconds or more, 45 seconds or more, 50 seconds or more, 55 seconds or more, 1 minute or more, 1.25 minutes or more, 1.5 minutes or more, 1.75 minutes or more, 2 minutes or more, 2.25 minutes or more, 2.5 minutes or more, 3 minutes or more, 3.5 minutes or more, 4 minutes or more, 4.5 minutes or more, 5 minutes or more, 5.5 minutes or more, 6 minutes or more, 7 minutes or more, 8 minutes or more, 9 minutes or more, 10 minutes or more, 15 minutes or more, 20 minutes or more, 25 minutes or more, 30 minutes or more, 35 minutes or more, 40 minutes or more, 45 minutes or more, 50 minutes or more, or 55 minutes or more). In some examples, the amount of time from placing the sample in the receptacle to output is 1 hour or less (e.g., 55 minutes or less, 50 minutes or less, 45 minutes or less, 40 minutes or less, 35 minutes or less, 30 minutes or less, 25 minutes or less, 20 minutes or less, 15 minutes or less, 10 minutes or less, 9 minutes or less, 8 minutes or less, 7 minutes or less, 6 minutes or less, 5.5 minutes or less, 5 minutes or less, 4.5 minutes or less, 4 minutes or less, 3.5 minutes or less, 3 minutes or less, 2.5 minutes or less, 2.25 minutes or less, 2 minutes or less, 1.75 minutes or less, 1.5 minutes or less, 1.25 minutes or less, 1 minute or less, 55 seconds or less, 50 seconds or less, 45 seconds or less, 40 seconds or less, 35 seconds or less, 30 seconds or less, 25 seconds or less, 20 seconds or less, 15 seconds or less, 10 seconds or less, 5 seconds or less, 4 seconds or less, 3 seconds or less, or 2 seconds or less). The amount of time from placing the sample in the receptacle to output can range from any of the minimum values described above to any of the maximum values described above. For example, the amount of time from placing the sample in the receptacle to output is from 1 second to 1 hour (e.g., from 1 second to 30 minutes, from 30 minutes to 1 hour, from 1 second to 1 minute, from 1 minute to 10 minutes, from 10 minutes to 1 hour, from 1 second to 55 minutes, from 1 second to 45 minutes, from 1 second to 30 minutes, from 1 second to 15 minutes, from 1 second to 10 minutes, from 1 second to 5 minutes from 1 second to 1 minute, from 5 seconds to 1 hour, from 10 seconds to 1 hour, from 15 seconds to 1 hour, from 30 seconds to 1 hour, from 45 seconds to 1 hour, from 1 minute to 1 hour, from 5 minutes to 1 hour, from 10 minutes to 1 hour, from 15 minutes to 1 hour, from 30 minutes to 1 hour, from 5 seconds to 55 minutes, or from 10 seconds to 45 minutes).

The devices comprise a computing device. Any of the methods disclosed herein can be carried out in whole or in part on one or more computing or processing devices.

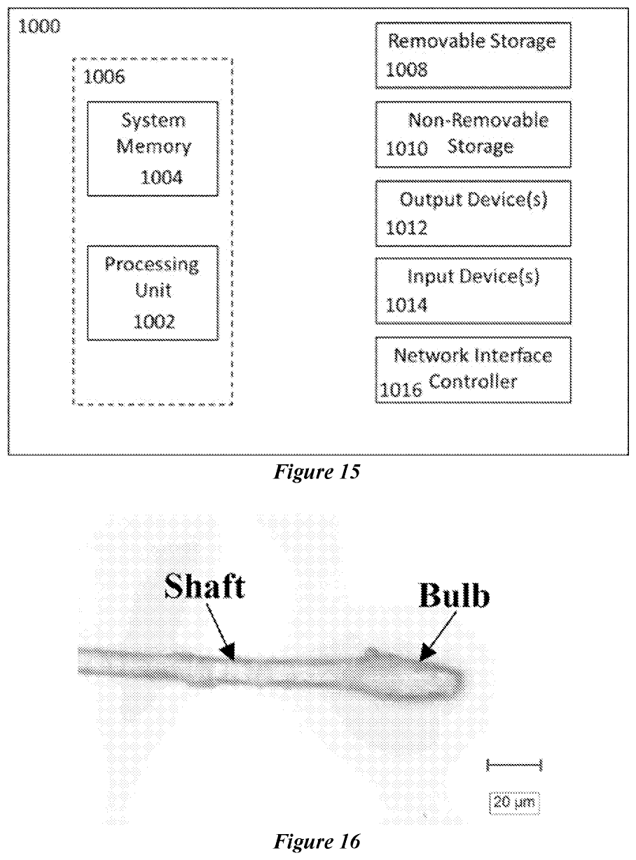

FIG. 15 illustrates an example computing device 1000 upon which examples disclosed herein may be implemented. The computing device 1000 can include a bus or other communication mechanism for communicating information among various components of the computing device 1000. In its most basic configuration, computing device 1000 typically includes at least one processing unit 1002 (a processor) and system memory 1004. Depending on the exact configuration and type of computing device, system memory 1004 may be volatile (such as random access memory (RAM)), non-volatile (such as read-only memory (ROM), flash memory, etc.), or some combination of the two. This most basic configuration is illustrated in FIG. 15 by a dashed line 1006. The processing unit 1002 may be a standard programmable processor that performs arithmetic and logic operations necessary for operation of the computing device 1000.

The computing device 1000 can have additional features/functionality. For example, computing device 1000 may include additional storage such as removable storage 1008 and non-removable storage 1010 including, but not limited to, magnetic or optical disks or tapes. The computing device 1000 can also contain network connection(s) 1016 that allow the device to communicate with other devices. The computing device 1000 can also have input device(s) 1014 such as a keyboard, mouse, touch screen, antenna or other systems configured to communicate with the camera in the system described above, etc. Output device(s) 1012 such as a display, speakers, printer, etc. may also be included. The additional devices can be connected to the bus in order to facilitate communication of data among the components of the computing device 1000.

The processing unit 1002 can be configured to execute program code encoded in tangible, computer-readable media. Computer-readable media refers to any media that is capable of providing data that causes the computing device 1000 (i.e., a machine) to operate in a particular fashion. Various computer-readable media can be utilized to provide instructions to the processing unit 1002 for execution. Common forms of computer-readable media include, for example, magnetic media, optical media, physical media, memory chips or cartridges, a carrier wave, or any other medium from which a computer can read. Example computer-readable media can include, but is not limited to, volatile media, non-volatile media, and transmission media. Volatile and non-volatile media can be implemented in any method or technology for storage of information such as computer readable instructions, data structures, program modules or other data and common forms are discussed in detail below. Transmission media can include coaxial cables, copper wires and/or fiber optic cables, as well as acoustic or light waves, such as those generated during radio-wave and infra-red data communication. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 1002 can execute program code stored in the system memory 1004. For example, the bus can carry data to the system memory 1004, from which the processing unit 1002 receives and executes instructions. The data received by the system memory 1004 can optionally be stored on the removable storage 1008 or the non-removable storage 1010 before or after execution by the processing unit 1002.

The computing device 1000 typically includes a variety of computer-readable media. Computer-readable media can be any available media that can be accessed by device 1000 and includes both volatile and non-volatile media, removable and non-removable media. Computer storage media include volatile and non-volatile, and removable and non-removable media implemented in any method or technology for storage of information such as computer readable instructions, data structures, program modules or other data. System memory 1004, removable storage 1008, and non-removable storage 1010 are all examples of computer storage media. Computer storage media include, but are not limited to, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by computing device 1000. Any such computer storage media can be part of computing device 1000.

It should be understood that the various techniques described herein can be implemented in connection with hardware or software or, where appropriate, with a combination thereof. Thus, the methods, systems, and associated signal processing of the presently disclosed subject matter, or certain aspects or portions thereof, can take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs can implement or utilize the processes described in connection with the presently disclosed subject matter, e.g., through the use of an application programming interface (API), reusable controls, or the like. Such programs can be implemented in a high level procedural or object-oriented programming language to communicate with a computer system. However, the program(s) can be implemented in assembly or machine language, if desired. In any case, the language can be a compiled or interpreted language and it may be combined with hardware implementations.

In certain examples, the methods can be carried out in whole or in part on a computing device 1000 comprising a processor 1002 and a memory 1004 operably coupled to the processor 1002, the memory 1004 having further computer-executable instructions stored thereon that, when executed by the processor 1002, cause the processor 1002 to carry out one or more of the method steps described above.

A number of embodiments of the invention have been described. Nevertheless, it will be understood that various modifications may be made without departing from the spirit and scope of the invention. Accordingly, other embodiments are within the scope of the following claims.

The examples below are intended to further illustrate certain aspects of the systems and methods described herein, and are not intended to limit the scope of the claims.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

Efforts have been made to ensure accuracy with respect to numbers (e.g., amounts, temperature, etc.) but some errors and deviations should be accounted for. Unless indicated otherwise, parts are parts by weight, temperature is in °C or is at ambient temperature, and pressure is at or near atmospheric. There are numerous variations and combinations of measurement conditions, e.g., component concentrations, temperatures, pressures and other measurement ranges and conditions that can be used to optimize the described process.

### Example 1—Noninvasive Raman Spectroscopy Assay for Radiation Exposure

Described herein are spectroscopic measurements for determining the exposure of an individual to ionizing radiation.

For example, Raman spectroscopy can be used to monitor changes in the chemical structure of hair originating from exposure to low dose ionizing radiation. Raman spectroscopy of hair provides a non-invasive measurement with the potential for tracking exposure events over a period of time. The Raman signal originates from the collective vibrations of molecules in the sample. Changes in the molecular structure result in changes to the intensity and frequency of the signal observed in the Raman spectrum. The Raman signal from the proteins in hair has previously been demonstrated to detect changes associated with hair color, drug use, and disease (Kumar P et al. Crit Rev Anal Chem 2021, 1-14; Pandey G et al. Forensic Sci Int 2017, 273, 53-63; Kurouski D et al. Anal Chem 2015, 87 (5), 2901-6; Pudney P D et al. Stanfield, S., Appl Spectrosc 2013, 67 (12), 1408-16; Galvan I et al. Spectrochimica acta. Part A, Molecular and biomolecular spectroscopy 2013, 110, 55-9; Zhang G et al. Journal of biomedical optics 2011, 16 (5), 056009: Wood J M et al. FASEB journal: official publication of the Federation of American Societies for Experimental Biology 2009, 23 (7), 2065-75; Ali E M et al. Anal Chim Acta 2008, 615 (1), 63-72; Gniadecka M et al. The Journal of investigative dermatology 1998, 110 (4), 393-8; Akhtar W et al. Spectrochimica acta. Part A, Molecular and biomolecular spectroscopy 1997, 53a (7), 1021-31; Schlucker S et al. Biopolymers 2006, 82 (6), 615-22).

Exposure to low dose ionizing radiation can impart changes to the hair present at the time of exposure that can persist in the hair shaft and be detectable from hair originating in the absence of radiation exposure.

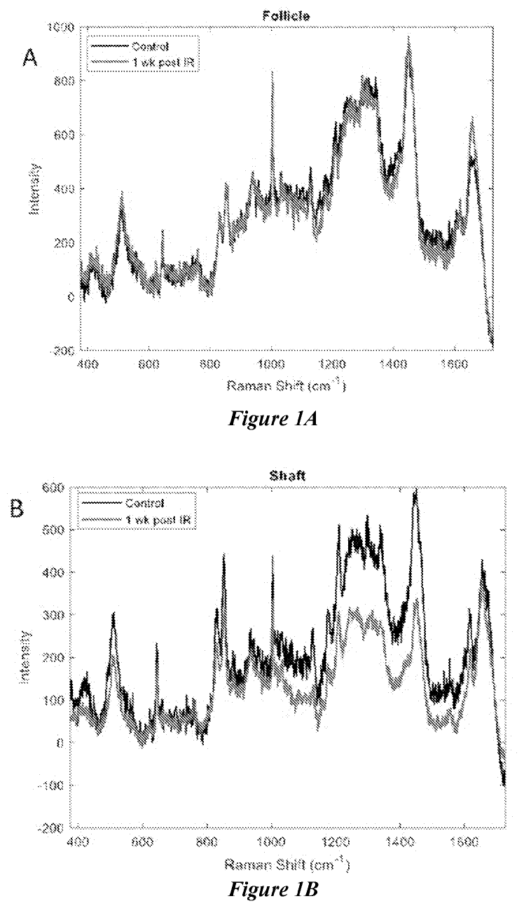

Data was acquired from hair samples of mice that were exposed to 14 Gy radiation (X-Rays), and unexposed mice. The hair from the irradiated mice was collected 1 week post irradiation. FIG. 1A-FIG. 1B show the average Raman spectra from biological and technical triplicates of hair samples, where the spectra were collected from the follicle and along the shaft of the hair. The spectra show bands consistent with the abundant keratin proteins found in hair. Of particular note, the spectra obtained from the hair follicles (FIG. 1A) of the irradiated and control mice are quite similar. The spectra show a slight difference in the amide I vibrations (c. 1650 cm⁻¹) (Peticolas W L. Methods Enzymol. 1995, 246, 389-416), which may suggest some change in the protein conformation between the two samples.

Striking differences are observed in the Raman spectra of the hair shaft between irradiated and control mice (FIG. 1B). Many spectral features are observed with lower intensity. Additionally, vibrational bands previously associated with functional groups that are expected to be altered by radiation exposure show changes in frequency and intensity that may be diagnostic for radiation dosimetry. In particular, the disulfide (—S—S—) spectral frequencies between 485-550 cm⁻¹ Raman shift show changes not observed in the hair follicles. Previous work has shown that changes in the conformation of the disulfide bond are detectable in the Raman spectrum (Sugeta H et al. Chem Lett 1972, 1, 83-86; Krimm S et al. In Advances in Protein Chemistry, Anfinsen C B et al. Academic Press: 1986, Vol. 38, pp 181-364; Sugeta H. Spectrochimica Acta Part A: Molecular Spectroscopy 1975, 31 (11), 1729-1737) and that disease conditions that disrupt the disulfide bonding are readily detectable (Schlucker S et al. Biopolymers 2006, 82 (6), 615-22). The disulfide linkages provide an attractive biomarker for radiation exposure, as they bonds are readily reduced, a likely outcome of exposure to ionizing radiation (Gerstel M et al. J Synchrotron Radiat 2015, 22 (2), 201-212; Bhattacharyya R et al. IUCrJ 2020, 7 (Pt 5). 825-834). Other amino acids that have been shown to be susceptible to ionizing radiation include aspartic and glutamic acid, which undergo decarboxylation (Gerstel M et al. J Synchrotron Radiat 2015, 22 (2), 201-212). Changes in these, and potentially other, amino acids provide a physical basis for the changes observed in the data. Metadata can be produced, for example decrease in disulfide bond intensity, that may have added value in conjunction with other markers of radiation exposure.

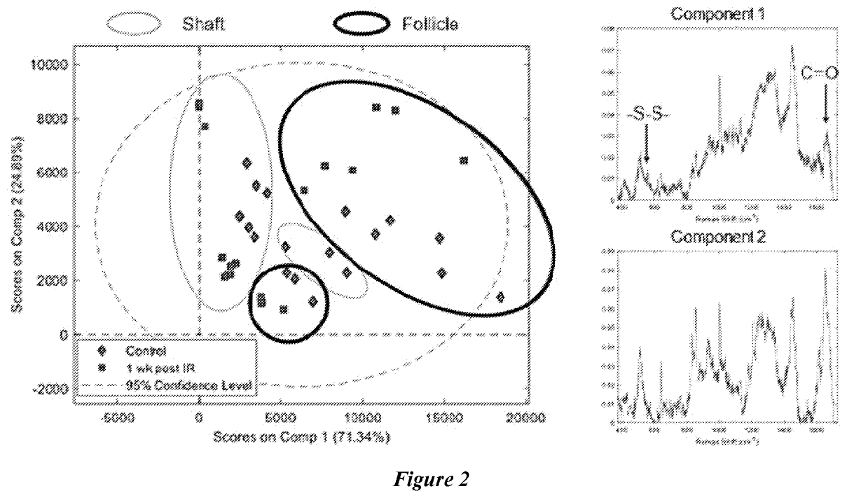

Multivariate analysis was performed to determine if the changes in the data appear to cluster based on exposure to ionizing radiation. FIG. 2 shows the multivariate curve resolution (MCR) analysis of the data collected from the various hair samples of irradiated and control mice. Component 1 shows a reduced number of vibrations that account for the variance in the data comparted to component 2. Component 2 resembles the Raman spectrum typically observed from protein samples. The analysis suggests that most of the variance in the data (71.34%) arises from changes associated with component 1. Of particular interest the vibrational frequencies associated with disulfide vibrations and carbonyl groups (blue arrows in component 1), the two groups readily affected by ionizing radiation (see above) show changes in this data. The PCA plot showing the scores of the samples on component 1 and component 2 show interesting clustering. Of particular importance, the irradiated hair shaft appears to cluster at low values of component 1, indicating the disruption of the disulfide and carbonyl bonds in these samples. Increased sampling, including number of animals, level of radiation exposure, and time from exposure, can enable a determination regarding whether radiation exposure can be quantified by the change in the observed Raman spectrum. Multivariate models can be analyzed for diagnostic capabilities in assessing exposure to ionizing radiation.

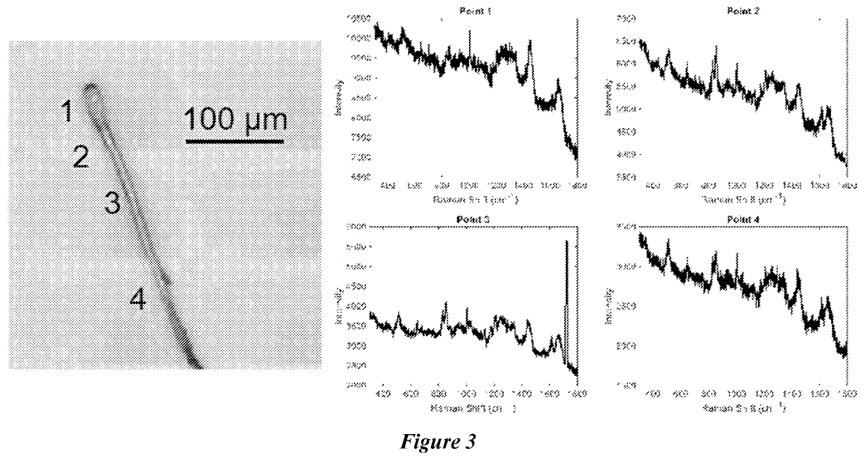

Analysis of hair samples suggests an interesting route to address changes in the sample. The data in FIG. 2 suggest that the follicles recover, or show no significant differences, from exposure to ionizing radiation. Additionally, the shaft of the control hair also shows a similar score on component 1 to the follicle data. Assessing the time since a radiation exposure event can be accomplished by using portions of the hair at different lengths. A Renishaw InVia Qontor microscope can mosaic visible images of the hair as shown in FIG. 3. While the tests herein explore changes at lengths of 100 μm, the microscope is capable to tiling images several inches in size (substantially larger than the mice hairs) to assess chemical composition from larger changes in hair growth. Mouse whiskers show a remarkable constant growth rate of approximately 1 mm/day (Ibrahim L et al. J Embryol Exp Morphol 1975, 33 (4), 831-44), which suggests an innate clock to assess time from radiation exposure. The rate of hair growth can be used to assess time, and Raman spectra at the appropriate lengths can be acquired to determine radiation exposure as a function of time.

The ability to monitor the part of the hair shaft associated with radiation exposure can be used to generate a straightforward metric of radiation exposure. A portion of the hair, associated with growth after radiation exposure or the follicle, can be ratioed to the Raman bands associated with the ionizing radiation. For example, the disulfide (—S—S—) band area in FIG. 1 can be ratioed against the corresponding spectroscopic band area of the follicle. This ratio can provide a quantitative metric of exposure. By assessing hairs at different levels of exposure, the observed chemical changes can be calibrated.

### Example 2

Described herein are baseline-independent biodosimetry assays that leverage highly sensitive and rapid analytical methodologies (e.g., Raman spectroscopy) and feature-based models to identify and quantify low dose radiological exposure events from noninvasive samples (hair and skin), unlike current methods that require baseline testing, and are non-sensitive and/or invasive. Rodent models are used first for discovery, characterization, and dose response analysis, which can then be validated in higher animal models and specimens from humans. The products can allow dose prediction and provide exposure characteristics under various exposure scenarios. The biomarkers' response can be used for dose and exposure characteristics reconstruction and evaluation without the need of a prior to exposure baseline reading. The products developed can have a sample to dose time of less than three hours. Acknowledging the complexity of the predicted exposure environment, this program is designed to deliver the products, integrating the impacts of radiation quality (electromagnetic, particulate type, and mixed exposure) and dose-rate, and time elapsed after exposure. The robustness and potential impact of confounders can be tested in multiple models and by utilizing other specimens.

Current radiation biodosimetry relies on clinical symptoms, lymphocyte depletion kinetics, and the dicentric chromosome assay (DCA) ((IAEA), I.A.E.A., EPR-biodosimetry, 2011. Vienna, Austria: IAEA; Dorr H et al., Radiat Res. 2017. 187(3): p. 273-286), which takes days to get readings. Studies over the last two decades have identified few panels of candidate radio-responsive serum proteins and messenger RNAs in peripheral blood; however, the responses observed were not robust across broader dose range ((IAEA), I.A.E.A., EPR-biodosimetry, 2011. Vienna, Austria: IAEA; Lucas J et al., PLoS One, 2014. 9(9): p. e107897; Ossetrova N I et al. Int J Radiat Biol, 2009. 85(10): p. 837-50). Translational utility of these findings with regard to sensitivity, accuracy, robustness, and rapidity are not evident, particularly after low dose exposure.

Radiation-induced changes in metabolites in body fluids, such as urine and blood, are transient and often have unfavorable kinetics, which is hypothesized to be similar also in interstitial fluids. For example, 2′-deoxyuridine (the deamination product of 2′-deoxycytidine) must be measured during a narrow, early window (6-12 hrs after radiation) (Tyburski J B et al., Radiat Res, 2009. 172(1): p. 42-57). Some metabolites and lipodomic markers alter as a function of dose, with utility at early time points (e.g., 24 h, 7 days); however, sensitivity is poor at lower dose range, and there are large variations in heterogeneous populations (Goudarzi M et al., Radiat Res, 2016. 186(3): p. 219-34; Pannkuk E L et al., Health Phys, 2018. 115(1): p. 3-11; Pannkuk E L et al., Sci Rep, 2017. 7(1): p. 9777: Pannkuk E. et al. Int J Radiat Biol, 2017. 93(10): p. 1151-1176). Meanwhile, the effects on rather solid tissues, such as hair and skin, are predicted to be long lasting.

Limit of detection and sensitivity at the lower range with the above biomarkers in body fluids is extremely challenging. In a Radiological/Nuclear scenario, the neutron exposure is expected in the order of 5-30% of the total dose, where the Relative Biological Effectiveness (RBE) is expected to be higher than photon exposure (Kramer K et al. Fort Belvoir, VA: Defense Threat Reduction agency; DTRA-TR-13-045 (R1); 2016, 2016), which could vary with pure versus mixed exposure (Cullings H M et al. Radiat Res, 2014. 182(6): p. 587-98). Furthermore, the impact of high-dose rate prompt exposure and relative low dose rate exposure from fallouts can be different, and the effect can be different from cumulative. All of these factors are considered herein.

Of note, a previously developed miR150-targeted finger-stick blood-based assay allows detection of dose at lower dose range (0.5 Gy resolution) without the need of baseline readings. Limitations include the fact that blood needs to be collected even though it is by finger-stick, signals are transient, and the ability for estimation is compromised after 7 days of exposure. However, with the knowledge and paradigm developed, biodosimeters can be developed using other analytical approaches such as Raman spectroscopy, using skin, hair root and hair shaft as non-invasive source materials. Raman spectroscopy can get an answer in ten minutes from a single hair and is particularly useful for obtaining information on elapsed time, factoring the metrics of hair growth.

A single model alone is unlikely to be enough to predict the dose and provide exposure characteristics, at early to late time points, particularly considering the complexity in the biology of response in a heterogeneous human population. Thus, feature-based models can be integrated and used. Normalization approaches developed during miR150-based biodosimetry can also be applied, which can allow dose prediction with information of exposure characteristics in a controlled manner without information on individuals' own prior baseline (Yadav M et al., Sci Transl Med, 2020. 12(552)).

The DOSIMETER (Discovery Of noninvaSive bloMarkEr for radiaTion ExposuRe) program is designed to discover, validate, and fine-tune dose response and dose reconstruction that allow automation and transition of the technology for various needs. The data can be used to develop advanced biodosimetry models and software tools to interpret biomarkers to identify exposure incidence, exposure dose, exposure dose-rate, exposure timeline, and other elements of the exposure incident. The product delivered from the assay can allow dose estimation within 10 min for Raman. The assay can be done using the equipment and resources available and cost per assay can be few dollars/assay.

The deliverables can include robust biomarkers and diagnostic assays that allow rapid and accurate detection of exposure of humans to ionizing radiation. To ensure delivery of products for dose prediction at both early as well as late time points, complementary analytical approaches can be employed. Both the raw and processed data generated for detection and characterization of biomarkers, generated in house and using provided T & E samples, and computational biodosimetry models can inform toward the dose, dose-rate, radiation type, and characteristics of real-life exposure scenarios.

Specifically:

Sample: Skin and hair—collected painlessly and unobtrusively. Specimens collected from rodents, and those used for validation in larger animals and humans.

Biodosimetry assays and models: The assay can be optimized and promising biomarkers that are unique or common across time points and exposure conditions can be discovered, characterized, and shortlisted, which can then be validated to ensure robustness of the identified signals. Sensitivity can be further developed by incorporating variables including dose types, dose-rate, and other exposure characteristics. Analytical pipelines can further be streamlined and optimized by considering confounding factors, specimens from humans, and integrating industry standards for diagnostics as needed for transitioning.

The end product can estimate dose or provide exposure characteristics, without the need of parallel analysis of baseline samples.

Raman spectroscopy-based radiation biodosimetry: Sample to dose: 10 minutes

Phase I A/B: Discovery/detection and characterization of biomarkers and optimize analytical methods: Mice (two strains) can be exposed to various doses (1-4 Gy gamma rays and 1-2 Gy neutron). Skin and hair specimens can be sampled on Day 1, 7, 25, 90, 120, and 180 in-house. Samples generated in-house and TE specimens can be used for discovery and characterization of biomarkers. Raman Spectrometry based analytical methods can be used for developing dose response curves. Round 1 and 2 TE evaluation.

### Milestones and Evaluation:

1. Assay optimized, characterize and short-list biomarkers in rodent model
2. Validate robustness in dose response in mice—two types of radiation (Gamma vs neutron)
3. Ensure the dose response in rodents and non-human primates samples (later available)
4. Early modeling, developed, and test responses in a blinded fashion (replicate)

Exit criteria: Responses detectable by ratiometrics, without baseline; At least one robust responder, detectable at two time points; down select the assay that do not meet the metrics

Phase IIA/B: Further characterization and validation of biomarker identified in Phase 1, focusing on sensitivity in dose range 0.1-0.75 Gy. Mice irradiation with varying dose rate (0.17, 3, 30, and 830 mGy/min) and sampling on Day 1, 7, 25, 90, 120, and 180: radiation types (photons, protons, neutrons, alpha particles, beta-particles, and photon-neutron mixed exposures).

Raman Spectrometry based analysis. Validate responses of the candidate biomarkers in humans and non-human primates: validation with modeling.

### Milestones and Evaluation:

1. Radiation quality effects, detect change with electromagnetic, particulate type vs mixed
2. Dose-rate effects; additive or not—signal ratios at various scenarios
3. Dose-response in humans, detectable at various time points and in non-human primates
4. Biodosimetry model developed and tested in a blinded fashion

Key exit criteria: Dose response and robustness vs. metrics

Phase III A/B: Advanced quantitation, testing, and validation of response in larger animal (porcine) model and test possible confounders such as UV, age/genetics/gender in mice and heterogeneous humans—(all races, pediatric, geriatric, chronic conditions, chemo pts). Advanced modeling, integration, and delivery of biodosimetry models

### Milestones and Evaluation

1. Validated the robustness of the biodosimeters
2. Assay time: optimization and transferability
3. Advance models developed following the guidelines

Deliverables: Robust biomarkers for non-invasive biodosimetry tested and validated in multiple models. Submit analytical protocols, models, and algorithms for estimating absorbed dose with information on dose-rate, time elapsed and exposure characteristics.

There is a critical need to develop rapid, reliable, deployable, and noninvasive diagnostic tools for retrospective detection (qualitative or semi-quantitative) and estimation of absorbed ionizing radiation dose (quantitative). Radiobiological studies established that dose time response mechanism after radiation exposure is extremely complex. Responses are mostly non-linear and without any defined threshold for specific biomarkers and injury response on sensitive organs and organ systems. Therefore, estimation of absorbed dose and analysis of exposure characteristics based on a single biomarker or model is unlikely to work across various time points. A programmatic approach can be used in animal studies, with regard to modeling radiation exposure types and scenarios. Considering the limitation of the ex vivo or 2D/3D constructs for dose estimation, especially at later time points, on biomarkers in animal models can be focused on and validated in human specimens.

Overview: A rodent (mouse) model can be used for discovery of biomarkers, early dose response analysis, and generating data for modeling, and as well as analytical methods. Raman spectroscopy, optionally in combination with other response-based approaches, can be used to investigate responses detectable in hair and skin that are likely the most exposed and accessible tissue types (non-invasive). The analysis can be reference tested. Animals can be irradiated and the specimens collected, and dose response and exposure characteristics can be modeled and tested at time points Day 1, 7, 25, 90, 120, and 180. The dose reconstruction algorithms developed using biomarker response in training groups can be validated using blinded samples.

In rodent studies, two days before irradiation, hair can be partly removed from a part of the body leaving 1-2 mm so that Raman Spectroscopy allows quantitative analysis over time with increasing length of the shaft. Mouse model studies for high dose radiation shows distinct kinetics and delayed responses in skin (Miller E D et al., Wound Repair Regen, 2019. 27(2): p. 139-149). Specimens from humans and higher animal models can be included, and robustness can be tested using baselines and various confounders. Some of these efforts in identifying and mitigating the confounders can be taken early for de-risking and timely transition. In the later phases of the project, these can be utilized to develop translatable assays, with existing diagnostic platform technologies. The archived specimens can be distributed for various analysis. Other specimens can be used for training, developing dose reconstruction algorithm, and blinded validation. Fast, easy-to-use, and end-to-end technologies can be developed for rapid delivery and transition.

Raman Spectroscopy-based biodosimetry. Raman spectroscopy can be used to monitor changes in the chemical structure of hair originating from exposure to low dose ionizing radiation. Raman spectroscopy of hair provides a non-invasive measurement with the potential for tracking exposure events over a period of time. The Raman signal originates from the collective vibrations of molecules in the sample. Changes in the molecular structure result in changes to the intensity and frequency of the signal observed in the Raman spectrum. The Raman signal from the proteins in hair has previously been demonstrated to detect changes associated with hair color, drug use, and disease (Kumar P et al., Crit Rev Anal Chem, 2021: p. 1-14; Pandey G et al., Forensic Sci Int, 2017. 273: p. 53-63; Kurouski D et al. Anal Chem, 2015. 87(5): p. 2901-6; Pudney P D et al., Appl Spectrosc, 2013. 67(12): p. 1408-16; Galvan I et al., Spectrochim Acta A Mol Biomol Spectrosc. 2013. 110: p. 55-9; Zhang G et al. J Biomed Opt, 2011. 16(5): p. 056009: Wood J M et al., Faseb j. 2009. 23(7): p. 2065-75: Ali E M et al., Anal Chim Acta, 2008. 615(1): p. 63-72; Gniadecka M et al., J Invest Dermatol, 1998. 110(4): p. 393-8; Akhtar W et al., Spectrochim Acta A Mol Biomol Spectrosc, 1997. 53a(7): p. 1021-31; Schlucker S et al., Biopolymers, 2006. 82(6): p. 615-22). Exposure to ionizing radiation can impart changes to the hair present at the time of exposure that can persist in the hair shaft and be detectable from hair originating in the absence of radiation exposure.

Data was acquired from hair samples of mice 1 week after 14 Gy (partial body) irradiation and unexposed mice as control. FIG. 1A and FIG. 1B show the average Raman spectra from biological and technical triplicates of hair samples, where the spectra were collected from the follicle and along the shaft of the hair. The spectra show bands consistent with the abundant keratin proteins found in hair. Of particular note, the spectra obtained from the hair follicles of the irradiated and control mice are quite similar. The spectra show a slight difference in the amide I vibrations (c. 1650 cm⁻¹) (Peticolas W L. Methods Enzymol., 1995. 246:p. 389-416), which may suggest some change in the protein conformation between the two samples.

Striking differences are observed in the Raman spectra of the hair shaft between irradiated and control mice (FIG. 1A and FIG. 1B). Many, but not all, spectra features are observed with lower intensity. Additionally, vibrational bands previously associated with functional groups that are expected to be altered by radiation exposure show changes in frequency and intensity that may be diagnostic for radiation dosimetry. In particular, the disulfide (—S—S—) spectral frequencies between 485-550 cm⁻¹ Raman shift show changes in hair shaft not observed in the follicles. Previous work has shown that changes in the conformation of the disulfide bond are detectable in the Raman spectrum (Sugeta H et al. Chemistry Letters, 1972. 1: p. 83-86: Krimm S et al. in Advances in Protein Chemistry, C. B. Anfinsen et al, Editors. 1986, Academic Press. p. 181-364: Sugeta H. Spectrochimica Acta Part A: Molecular Spectroscopy, 1975. 31(11): p. 1729-1737) and disease conditions that disrupt the disulfide bonding are readily detectable (Schlucker S et al., Biopolymers, 2006. 82(6): p. 615-22). The disulfide linkages provide an attractive biomarker for radiation exposure, as these bonds are readily reduced, a likely outcome of exposure to ionizing radiation (Gerstel M et al. Journal of synchrotron radiation, 2015. 22(2): p. 201-212; Bhattacharvya R et al., IUCrJ, 2020. 7(Pt 5): p. 825-834). Other amino acids that have been shown to be susceptible to ionizing radiation include aspartic and glutamic acid, which undergo decarboxylation (Gerstel M et al. Journal of synchrotron radiation, 2015. 22(2): p. 201-212). Changes in these, and potentially other, amino acids provide a physical basis for changes observed in the data. Metadata can be produced, for example, decrease in disulfide bond intensity that may have added value in conjunction with other markers of radiation exposure.

Multivariate analysis was performed to determine if the changes in the data appear to cluster based on exposure to ionizing radiation. FIG. 2 shows the multivariate curve resolution (MCR) analysis of the data collected from the various hair samples of irradiated and control mice. Component 1 shows a reduced number of vibrations that account for the variance in the data comparted to component 2. Component 2 resembles the Raman spectrum typically observed from protein samples. The analysis suggests that at least a portion of the variance in the data arises from changes associated with component 1. Of particular interest, the vibrational frequencies associated with disulfide and carbonyl vibrations (indicated by the blue arrows pointing to component 1 in FIG. 2) show changes in the data.

The MCR plot showing the scores of the samples on component 1 and component 2 show interesting clustering. Of particular importance, the irradiated hair shaft appears to cluster at low values of component 1, indicating the disruption of the disulfide and carbonyl bonds in these samples. Increased sampling, including number of animals, level of radiation exposure, and time from exposure, can enable a determination regarding whether radiation exposure can be quantified by the change in the observed Raman spectrum. Multivariate models can be analyzed for diagnostic capabilities in assessing exposure to ionizing radiation.

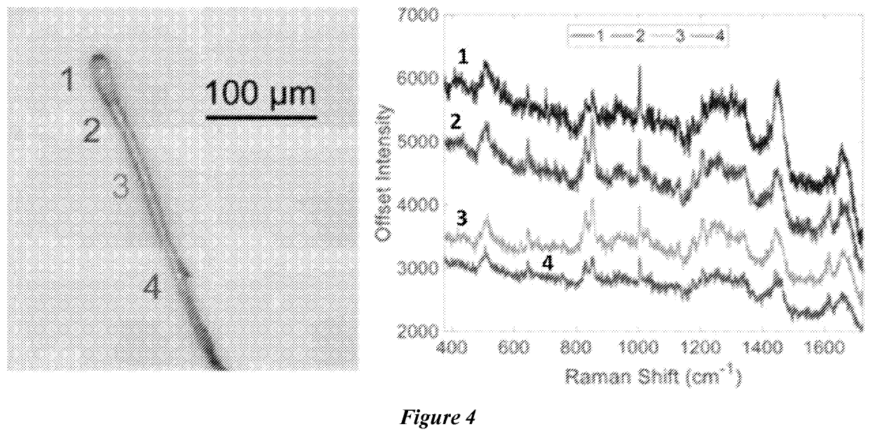

Analysis of hair samples suggests an interesting route to address changes in the sample. The data in FIG. 4 suggest that the follicles recover, or show no significant differences, from exposure to ionizing radiation. Additionally, the shaft of the control hair also shows a similar score on component 1 to the follicle data. Portions of the hair at different lengths can be used to assess time since radiation exposure event. A Renishaw InVia Qontor microscope can mosaic visible images of the hair as shown in FIG. 4. While these tests explore changes at lengths of 100 μm, the microscope is capable of tiling images several inches in size (substantially larger than the mice hairs) to assess chemical composition from larger changes in hair growth. Mouse whiskers show a remarkable constant growth rate of approximately 1 mm/day (Ibrahim L et al. J Embryol Exp Morphol, 1975. 33(4): p. 831-44), which suggests an innate clock to assess time from radiation exposure. The rate of hair growth can be Raman spectra at the appropriate lengths can be acquired to determine radiation exposure as a function of time.

Raman Spectroscopy and Analysis: The ability to monitor the part of the hair shaft associated with radiation exposure can be used to generate a straightforward metric of radiation exposure. A portion of the hair, associated with growth after radiation exposure or the follicle, can be ratioed to the Raman bands associated with the ionizing radiation measured from hair existing during the radiation event. For example, the disulfide (—S—S—) band area from the shaft in FIG. 4 can be ratioed against the corresponding spectroscopic band area of the follicle. This ratio can provide a quantitative metric of exposure. By assessing hairs at different levels of exposure, the observed chemical changes can be calibrated. These can provide quantitative metrics for dose response, exposure duration, and radiation type (electromagnetic vs particulate) dependent response.

Changes in Raman bands attributable to radiation exposure can provide a ratiometric model to quantitatively determine the exposure dose. Studies in Phase I can demonstrate feasibility of the method for dose prediction, determining duration and type of exposure. Phase II can demonstrate feasibility of the method in response to dose rate, duration, and type of radiation. Phase III can further validate responses in larger animal models and can test the effect of various confounders.

Confounders and possible interferents for the Raman analysis include changes in hair color and the presence of exogenous compounds (e.g. shampoo and conditioner residues). Animal samples can be washed (acetone+water) to remove exogenous compounds. Human samples collected can be from cosmetically untreated hair (Eisenbeiss L et al. Anal Bioanal Chem, 2019. 411(17): p. 3963-3977). Tests can be performed on both colored (dark: brown/black) hairs and compared with white and gray hairs (e.g. mice C57BL/6-black and BALB/c-white). The increased pigment levels in colored hair may exhibit fluorescent backgrounds from pigments. Chemical or photo-bleaching approaches can be performed to minimize the fluorescence interference from pigments, if needed. The Raman microscope is equipped with multiple excitation lasers that can be employed to minimize background fluorescence. Preliminary tests on gray hair were not affected by a fluorescent background. To account for differences in color and the presence of exogenous compounds, Raman analysis can be performed on different sections of the hair shaft.

Data suggests the hairs' chemical composition recovers post exposure, which allows for portions of the hair sample to be compared to itself for quantitative analysis. Hair follicles provide an additional control for the chemical changes being monitored in exposed hair samples.

There are challenges in normalization and translating the response to a dose, with a single sampling, without information on the baseline. However, applying the internal normalization technique employed in Yadav M et al., can help reduce the uncertainty of the absorbed dose measurement (Yadav M et al., Sci Transl Med, 2020. 12(552)). Such an approach could be further extended with the help of artificial intelligence. First, small data sample sets from animals can be used to train the model, and then the model can be applied on unknown data sample sets to predict the absorbed dose and validate the model through the comparison of the predicted dose with the model measured dose.

General statistics: Prior to conducting advanced modeling to select biomarkers, statistical tests can be performed to identify molecules (e.g., peptides, amino acids) that respond to exposure. Analysis of variance (ANOVA) can be used for rat/human data and ANOVA with repeated measures can be used for the porcine experiment accounting for observational dependencies to obtain the list of candidate biomarkers. Type I error rate can be controlled by false discovery rate (FDR). Molecules at very low levels across conditions can be first removed to minimize false negative rate. Data normality can be checked and transformations such as nature log can be applied if necessary. Molecules identified can be served as starting candidates for model selection using LASSO method mentioned above or nonparametric approach such as random forest. Specificity and sensitivity analysis can also be conducted to evaluate the predictability of exposure of the selected biomarkers. Additionally, through data display, such as heatmaps, the molecules remain unchanged after exposure with different doses, and time can be selected and serve as an internal matched control for future panel tool development.

Risk mitigation: One of the major challenges in the later phases can be in the sensitivity of the biomarker and biomarker assays at lower dose ranges (0.1-0.75 Gy) without significant influence of the confounders. Testing the background reading in diversity outbred mice, including those from both genders, age group, and other potential confounders, comparison of available specimens from non-human primates and heterogeneous can allow robustness to be validated. The use of technical as well as biological replicates can be included at both the testing and validation. The assay that reports numerical results can provide detailed information on interval studies, with controls and details including limit of detection, and limit of quantification and details on the evaluation of detection capability.

Qualitative risk assessment involves identifying the probability and impact of possible scenarios involving the cost, schedule, and sustainability of the project. Risks can be mitigated by including time buffers within the experimental design to ensure timely completion. During the animal studies and the data collection, the potential risks in the project involve delays of assay supplies and animal delivery.

Deliverables. The program includes testing biomarkers in a highly integrated manner, enhancing the chance for success and delivery of a possibly comprehensive biodosimetry solution with single sampling that is practical for various time points and scenarios. The combinatorial approaches can allow robust biodosimetry, days to months or even years after exposure.

Raman spectroscopy based rapid biodosimetry—sample to dose time~10 minutes

Validation can occur for each product component and then as a complete system: 1) The assay 2) instrumentation and 3) Software or software/instrument interface and functionalities. Protocols and workflows for biomarker discovery and characterization associated raw as well as processed data.

### Example 3—Noninvasive Raman Spectroscopy Assay for Radiation Exposure

Ionizing radiation is a form of energy that can pass through materials such as air, water, and living tissue. It is distinguished from non-ionizing radiation in that it removes electrons from atoms or molecules of material and can cause skin or tissue damage and eventual harm such as cancer. The health effects of radiation depend on the type, duration of exposure, the amount of radiation generated from the source, distance from the source, and the amount and type of shielding of the individual. Radiation is measured in interrelated units that measure radioactivity, exposure, absorbed dose, and dose equivalent. Radioactivity is the amount of ionizing radiation released by a material in Curie (Ci) and becquerel (Bq) units, whereas radiation exposure is measured in roentgen (R) and coulomb/kilogram (C/kg) and measure the amount of radiation traveling through the air.

Radiation biodosimetry determines a past radiation dose from an exposure incident and is distinguished from a bioassay, which determines past, current, and future radiation dose from a contamination incident. Radiation biodosimetry is a method for estimating exposure to an individual and the current gold standard method is dicentric chromosome assay (DCA). DCA dose prediction is “based on ionizing radiation-induced damage to DNA, which results in the formation of dicentric chromosomal aberrations. The number of dicentric chromosomes increases with the amount of radiation allowing estimation of unknown dose.” However, DCA is not well suited for mass screening, requires a high level of technical input, low throughput, and has a specific time window for use. Point of care methods for biodosimetry include lymphocyte depletion kinetics and clinical exam: however, the time window presents a limiting factor, as does the requirement for an early lymphocyte count to establish a baseline.

Radiation biomarkers and other advanced methods are currently being studied using various methods. The methods described herein combine different models to quantify radiological exposure without the need for a baseline and leverages noninvasive biological samples such as skin and hair.

The technology comprises a spectroscopic measurement to determine the exposure of an individual to ionizing radiation. The technology leverages highly sensitive and rapid analytical methodologies (Raman spectroscopy) and feature-based models to identify and quantify low dose radiological exposure events from noninvasive samples (hair and skin), unlike current methods that require baseline testing and are non-sensitive and/or invasive. Preliminary testing has been conducted in rodents. The technology can allow for dose prediction and provide exposure characteristics under various exposure scenarios. The biomarkers' response can be used for dose and exposure characteristics reconstruction and evaluation without the need of a prior to exposure baseline reading. The products can have a sample to dose time of less than three hours.

Raman spectroscopy can monitor changes in the chemical structure of hair originating from exposure to low-dose ionizing radiation. Raman spectroscopy of hair provides a noninvasive measurement with the potential for tracking exposure events over a period of time. The Raman signal originates from the collective vibrations of molecules in the sample. Changes in the molecular structure result in changes to the intensity and frequency of the signal observed in the Raman spectrum.

The proposed technology suggests a method for leveraging robust biomarkers and diagnostics assays that allow rapid and accurate detection of ionizing radiation exposure in humans in a noninvasive manner using skin and hair. The technology is a tool leveraging Raman spectroscopy for determining radiation exposure. Further development of initial animal studies can generate a working prototype with a reproducible and reliable method.

The global dosimeter market was valued at $2.686 billion in 2020 and is expected to grow at a CAGR of 7.68% to reach an estimated $4.214 billion by 2026. The market has been impacted by the COVID-19 pandemic through challenges faced by technical services in radiation protection and safety. Despite this, the market is witnessing growth through increasing demand in the medical industry and industrial sites, use in cancer treatments, management of hazardous waste or radioactive substances, and radiation protection aims. The market is also expected to witness growth in the development of nuclear energy and facilities for industrial work. Regionally, Europe comprises the largest market share based on the increasing prevalence of cancer and the use of radiation therapies and nuclear medicines, thereby driving the use of dosimeters.

### Example 4

Data was acquired from hair samples of mice that were exposed to radiation, and unexposed mice. Hair samples were analyzed using a Renishaw InVia Qontor microscope with a 50× objective. For the analysis, hair was placed on gold coated slide and data was collected using 10% laser power (785 nm with pinhole) (samples burned at higher laser power). Data was collected using 60 second acquisitions; 3 spectra were collected per spot. Spectra were taken from various points of the hair; 3 hairs per sample were used. Control and one week post irradiation (14 Gy WTLI, Whole Thorax Lung Irradiation) samples were compared using MCR.

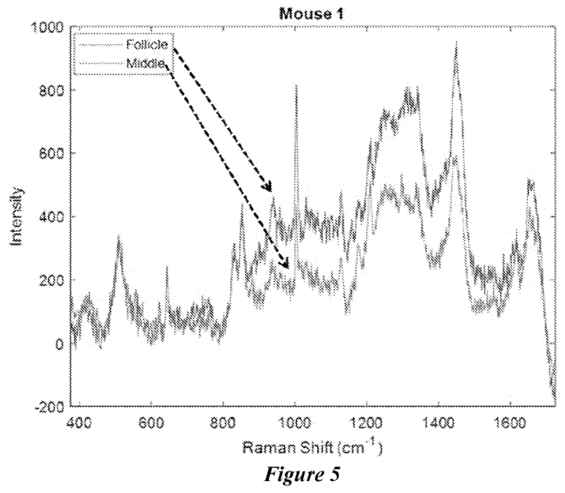

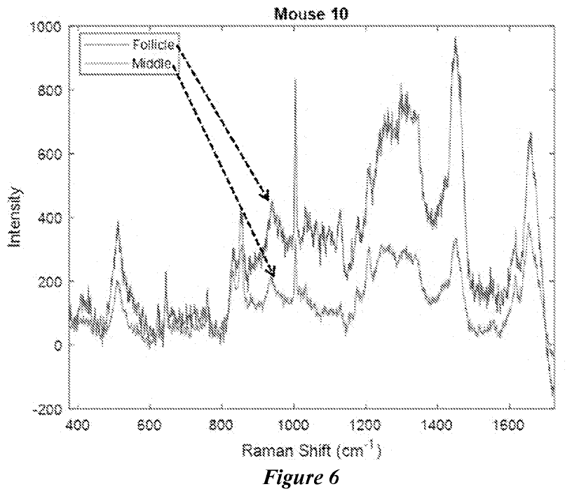

Example spectra collected at different points along the hair for mice (average of 9 spectra per sample) are shown in FIG. 5 and FIG. 6.

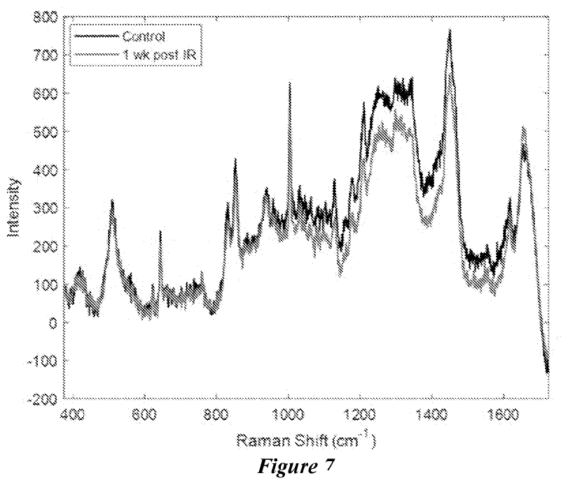

Example spectra for control and one week post irradiation (14 Gy WTLI) samples (average of 18 spectra per sample) are shown in FIG. 7.

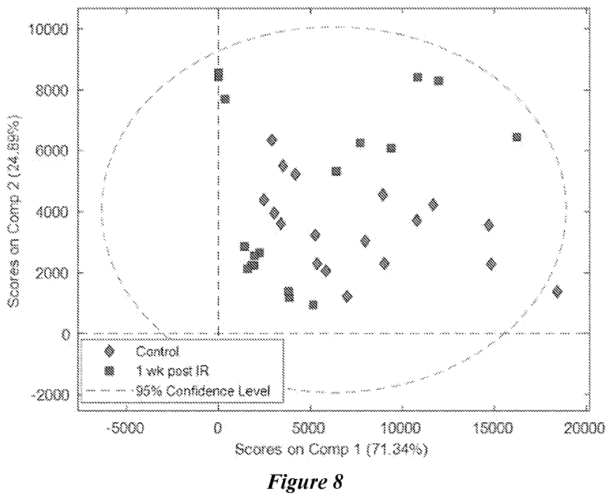

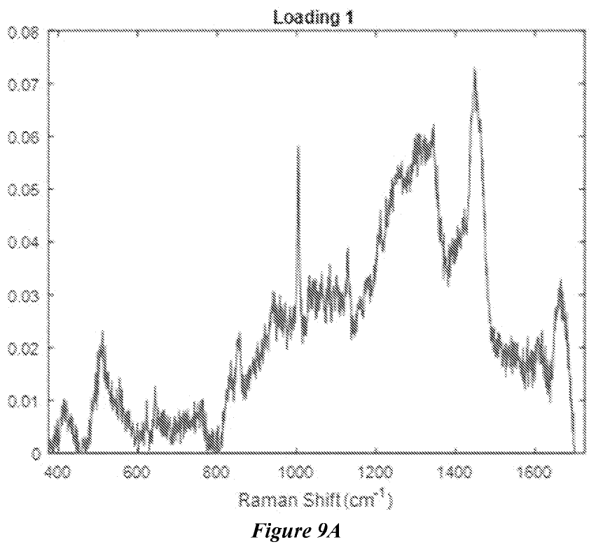

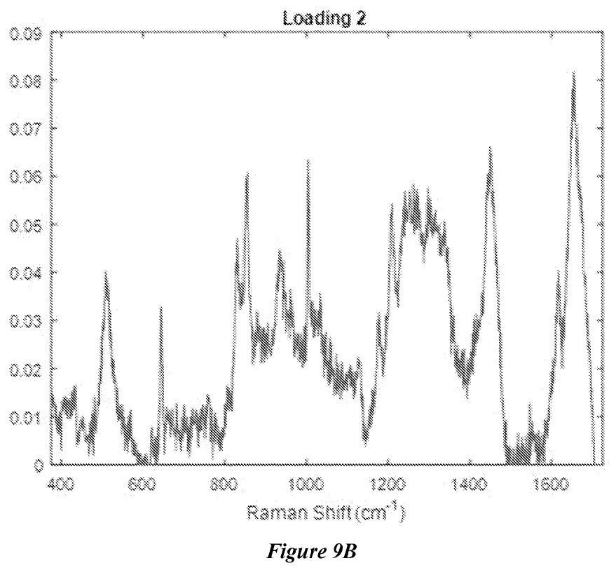

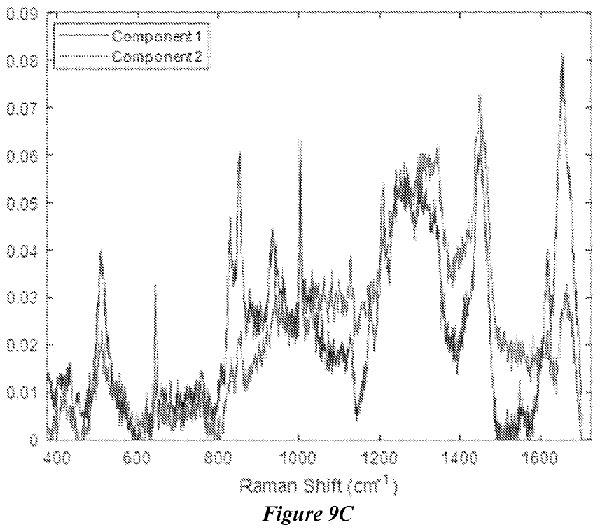

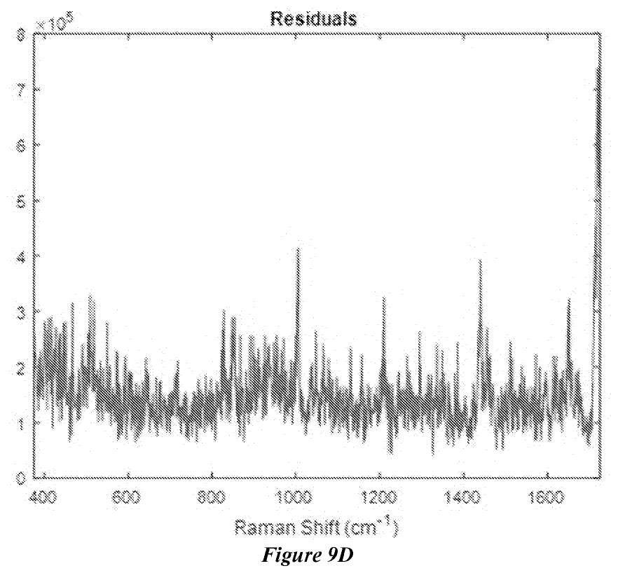

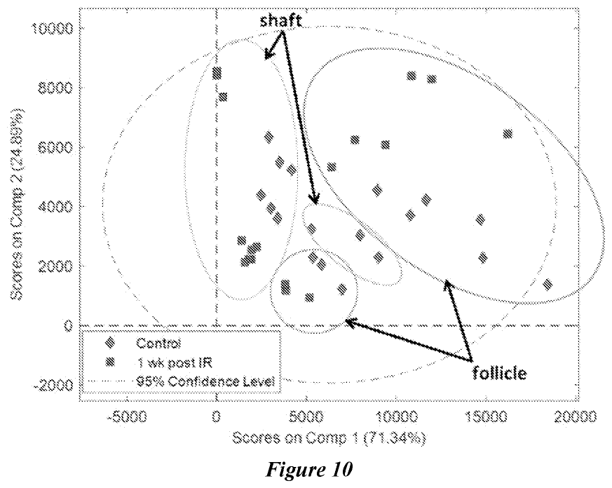

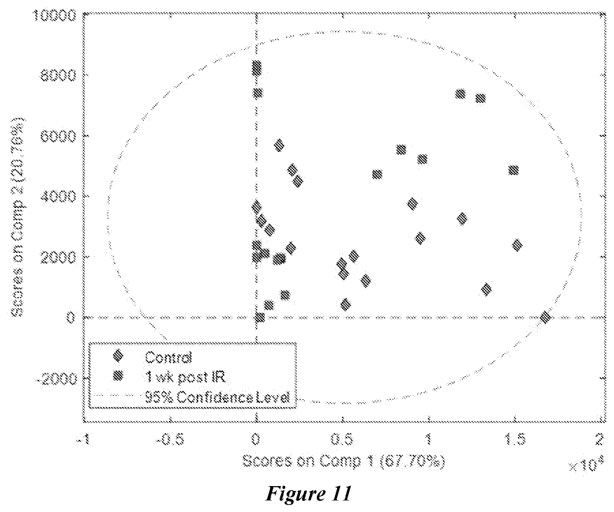

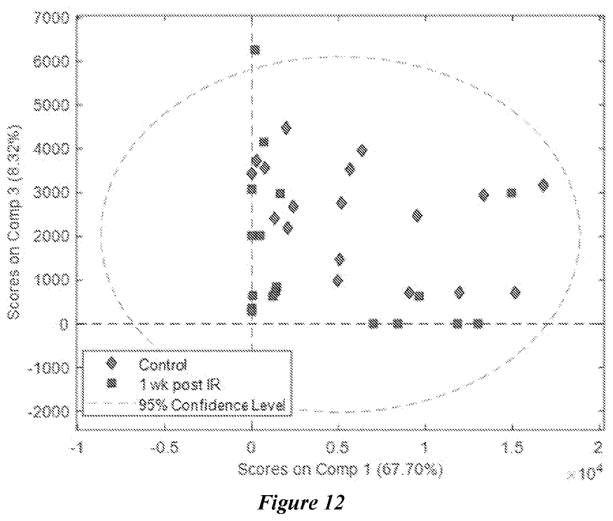

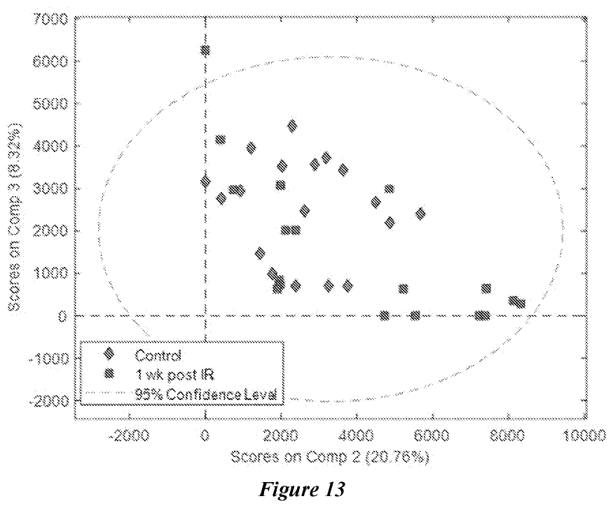

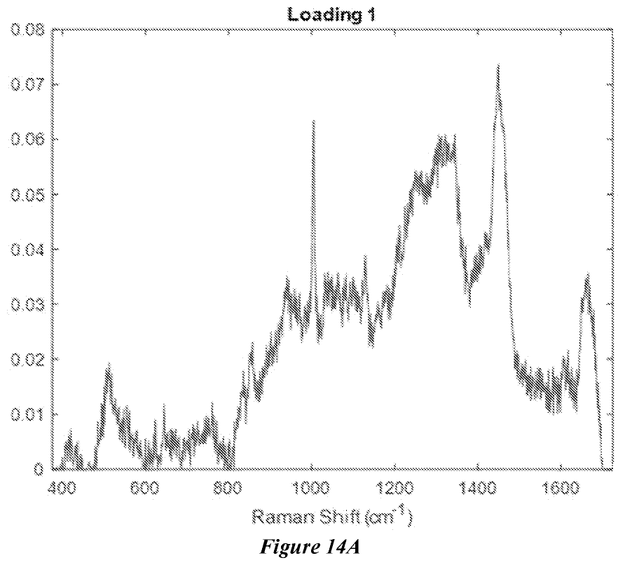

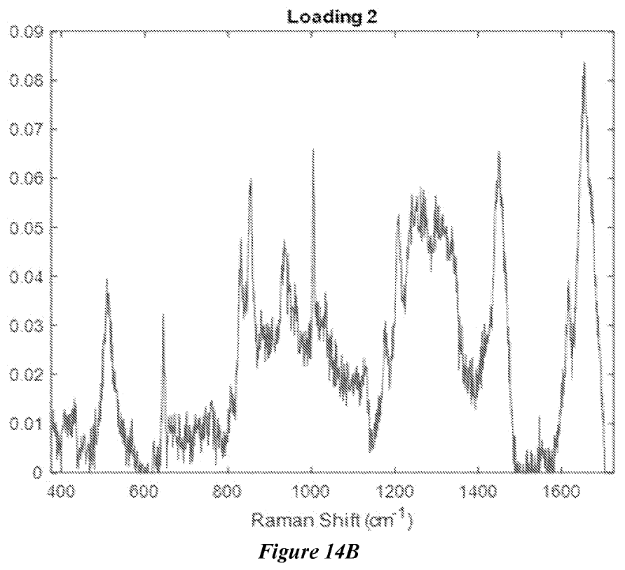

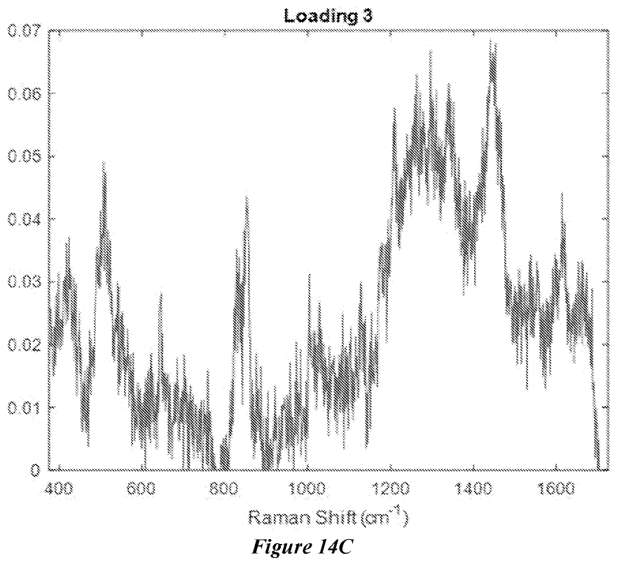

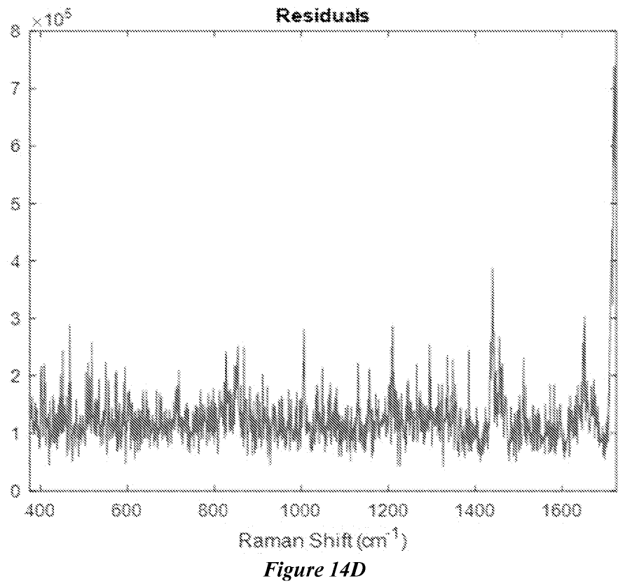

Control and one week post irradiation (14 Gy WTLI) samples were compared using MCR. The results for a 2 component MCR model are shown in FIG. 8-FIG. 10. The results for a 3 component MCR model are shown in FIG. 11-FIG. 14D.

### Example 5

Exposure to radiation through unintentional contact with radioactive material (e.g., natural environmental or occupational settings) or through intentional release of a radioactive material has major health consequences. The radioactive contamination released into the environment can comprise radionuclides that emit alpha, beta, gamma radiation, neutrons, or a combination with varying hazard levels. Some exposures may not present visible clinical signs, especially at lower dose range, yet have significant delayed consequences, and early detection and risk assessment would allow timely mitigation. As of now, there is no non-invasive biodosimetry assay or method approved for human use.

Disclosed herein are rapid and accurate biodosimetry solutions, based on biomarkers detectable in skin and hair. The methods involve radiation biodosimetry, biomarker discovery, and validation using state of the art analytic platforms such as Raman spectroscopy, data science and artificial intelligence-assisted modeling.

Cohorts of mice can be irradiated at the various doses. Skin and hair sampled at Day 1, 7, 25, 90, and 120 can be collected, and subjected to Raman spectroscopy-based biomarker discovery for obtaining quantitative and qualitative information on exposure dose, duration, radiation types, and dose-rate effects. Shortlisted candidates can be validated further in larger animal models, including samples from control as well irradiated non-human primates, domestic pigs, and human samples. These can become the basis for development of end-to-end use diagnostic assays with integrated software and dose reconstruction and prediction.

Major strengths of these methods include complementary approaches, multi-modal modeling of exposure scenarios, and reference-controlled assays for predicting the dose from a single sample of unknown exposure without the need of prior baseline sampling. Data presented here suggests Raman spectroscopy is capable of estimating time elapsed since exposure from a single hair strand. The characteristics tested can include readability even with intermittent exposure, considering the scenarios of unknown exposure soldiers and civilians may come across. The combinatorial approaches can allow robust biodosimetry, days to months or even years after exposure. Acknowledging the complexity expected in prediction of low dose in a heterogeneous population, potential confounding factors including age, gender, race, and underlying conditions have been considered. Deliverables include robust biodosimetry solutions, with information on exposure characteristics across varying time points, with a sample to dose time of 10 min (Raman spectrometry). These can have a significant impact as these are predicted to be sensitive, accurate, and can provide exposure characteristics.

### Example 6—Protocol for Sample Handling, Pigment Data Collection, and Processing

### Sample Handling and Preparation

Sample Storage. Samples are stored in −80 °C freezer before and after Raman analysis. Samples are removed from the freezer at least 30 minutes before Raman analysis to allow for thawing, and the un-aliquoted samples are replaced after analysis.

Sample Preparation. Once thawed, a small aliquot (approximately 3-10 strands) of hairs are transferred to double sided tape on a gold coated microscope slide. The metal coating prevents glass interference and increases the Raman signal. The double-sided tape ensures the hairs are flat on the slide. Gold slides are rinsed with absolute ethanol and dried with a Kimwipe (lint free cleaning tissue), and a new piece of double-sided tape is applied between hair samples.

### Instrumentation and Startup

Instrumentation. Data collection is performed on a Renishaw inVia Qontor Microscope (Renishaw plc). Raman excitation source is a 785 nm diode laser (Renishaw plc) and a 532 nm diode laser (Renishaw plc). Laser light is focused on samples through a Leica N PLAN 50×L, 0.50 NA BD objective. Backscattered light from the 785 nm laser is collected by the same objective, sent through a Rayleigh filter and diffracted by a 1200 lines/mm grating. Backscattered light from the 532 nm laser is collected by the same objective, sent through a Rayleigh filter and diffracted by an 1800 lines/mm grating. Diffracted light is sent to a −70 °C (thermoelectrically cooled), 1024×256 pixel, Charge coupled device (CCD) detector.

Renishaw startup procedure—785 nm excitation. The laser is allowed to warm up for at least 30 minutes before alignment for pointing and stability.

inVia auto-alignment procedures are performed in the following order as recommended by the instrument manufacturer: Silicon standard position; Laser alignment which optimizes the beamsteer motors; CCD confocal area which optimizes the area on the detector; The slit positions are optimized going into the spectrometer.

The calibration is corrected for drift by performing quick calibration which offsets the calibration, so the silicon phonon lands at 520.5 cm⁻¹.

785 nm laser performance Checks. Laser power is measured with a Thorlabs S130C slim power sensor. The power sensor is connected to a Thorlabs PM100A power meter console with mechanical and graphical displays. After alignment, the laser power at the sample is measured for the settings 100%, pinhole out and logged. On this system, this is around 100 mW at optimal performance. The laser power for data collection (10%, pinhole in) is also measured. This can be used to correct detector counts for fluctuations in laser power. The laser power is around 0.7 mW at the sample.

Renishaw startup procedure—532 nm excitation. The laser is allowed to warm up for at least 30 minutes before alignment for pointing and stability.

inVia auto-alignment procedures are performed in the following order as recommended by the instrument manufacturer: Silicon standard position; Laser alignment which optimizes the beamsteer motors; CCD confocal area which optimizes the area on the detector; The slit positions are optimized going into the spectrometer.

The calibration is corrected for drift by performing quick calibration which offsets the calibration, so the silicon phonon lands at 520.5 cm⁻¹.

532 nm laser performance Checks. Laser power is measured with a Thorlabs S130C slim power sensor. The power sensor is connected to a Thorlabs PM100A power meter console with mechanical and graphical displays. After alignment, the laser power at the sample is measured for the settings 100%, pinhole out and logged. On this system, this is around 16 mW at optimal performance. The laser power for data collection (1%, pinhole in) is also measured. This can be used to correct detector counts for fluctuations in laser power. The laser power is around 0.1 mW at the sample.

### Data Collection

Frequency calibration check standard Acetaminophen is a published standard (ASTM E 1840) (Standard Guide for Raman Shift Standards Spectrometer Calibration. In Molecular Spectroscopy and Separation Science; Surface Analysis; Vol. ASTM 03.06). To check the calibration of the Renishaw Qontor, an acetaminophen sample is run at the start of each analysis day after startup. A high power (50% ND, pinhole out), 10 second exposure, extended scan (100-3200 cm⁻¹) is performed at both 785 nm and 532 nm. In the WiRE software, each peak (in the fingerprint region 300-1800 cm⁻¹) is fitted and the frequencies are ensured to be within ±1 cm⁻¹ of their published values.

Hair Raman collection parameters—785 & 532 nm excitation. Scan type: Extended, over 200-3200 cm⁻¹. Exposure time: 10 seconds. Single acquisition. Laser power: 532 nm=0.1 mW; 785 nm=0.7 mW. White light images are acquired with the inVia camera before and after spectral acquisition. Detector confocality: high. Pinhole in.

### Sampling Parameters

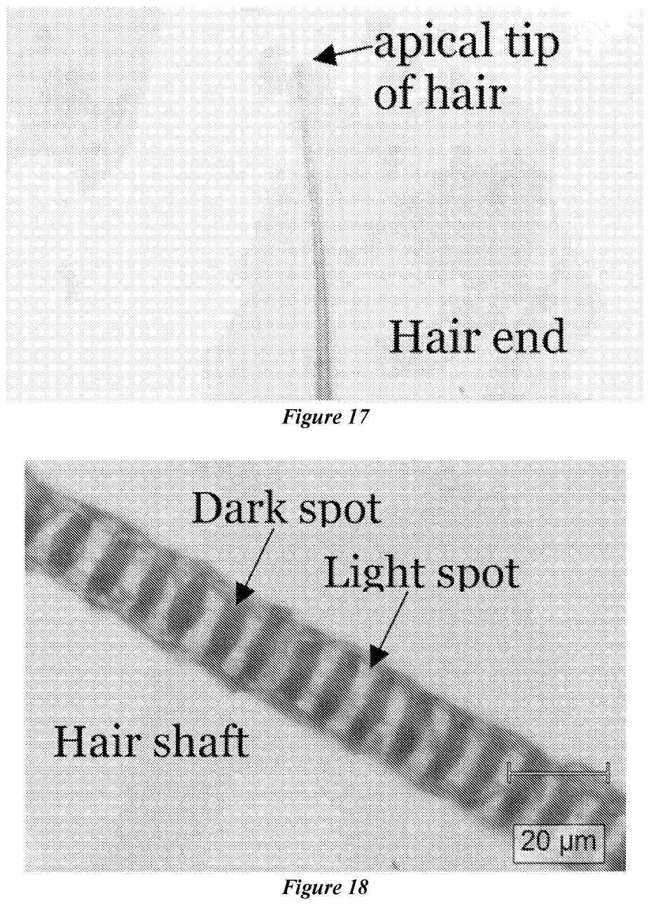

Samples are found in the microscope that are fully intact, having both a root bulb (FIG. 16) and a full hair end (FIG. 17). Focus is found on the sample in a spot that is clear of damage. The stage coordinates of the very end tip of the hair as well as inflection points along the hair are also recorded. For each mouse, three hairs data are collected as technical replicates.

Root bulb. Focus is found on the root bulb and that spot is set to be the origin of the stage. Data is first collected with the 532 nm excitation laser and then with the 785 nm excitation laser.

Light Spots. The laser is moved about 1 mm along the hair from the last spot measured to a spot free of damage. The laser spot is centered and focused on a light spot first (FIG. 18). Data is collected with the 785 nm excitation laser and then with the 532 nm excitation laser. These measurements are both taken at the same spot, the stage coordinates of that spot are recorded. If no light spot is observed, then only a dark spot is measured.

DarkSpots. The laser spot is centered and focused on an adjacent dark spot (FIG. 18). Data is first collected with the 532 nm excitation laser and then with the 785 nm excitation laser. These measurements are both taken at the same spot, the stage coordinates of that spot are recorded. If there is any visible photo damage to the hair, a new dark spot is used instead. This spot is far enough away to not be damaged while still being close to the light spot. If this cannot be achieved, an entire new spot clear of damage is used for both the light and dark spots.

Ends. Focus is found at the end of the hair on a spot free of damage. Data is first collected with the 532 nm excitation laser and then with the 785 nm excitation laser. These measurements are both taken at the same spot, the stage coordinates of that spot are recorded. If there is any visible photo damage to the hair, a new spot is used instead.

### Data Processing

Software (WiRE™ 5) preprocessing. Cosmic rays are removed in WiRE™ 5 using the width of features algorithm. Cosmic ray-free files are saved as a separate .wdf file. Using the Batch File Converter (proprietary with WiRE™), cosmic ray-free files are converted to .txt for import into Matlab (Mathworks®).

Matlab preprocessing and analysis. Spectral data are imported into Matlab. The Raman shift values at each pixel are kept with each dataset for feature extraction. Data is baseline corrected using a rolling circle background subtraction code with a radius of 1,000,000 (Brandt et al. Optimization of the Rolling-Circle Filter for Raman Background Subtraction, Appl. Spectrosc. 2006, 60, 288-293). Data is normalized to the power, by dividing the data by the power of the laser it was measured with. The 785 nm data is also shortened to a Raman shift range of 3146.6-279.6 cm⁻¹ in order to cut off the ends that were not properly background subtracted by the rolling circle filter.

Mouse hair length analysis. The x, v, and z coordinates of each hair are imputed into an excel sheet. The distance formula is used to determine the distance between individual points along the hair in sequential order. These are added in a way to find the distance from the follicle for each data point as well as the total length of the hair.

dist.=\sqrt{(X₁−X₂)²+(Y₁−Y₂)²+(Z₁−Z₂)²}

This was done separately for the dark and light spots because some movements from the light to dark spots are in the direction of the width and not the length of the hair.

### Feature Extraction and Data Delivery

Feature extraction. Curve fitting analysis with Peakfit 9.0 (Matlab program) is performed to extract intensity values from spectra in the 400-1800 cm⁻¹ region. A distribution of the program can be acquired from the MATLAB file exchange (https://www.mathworks.com/matlabcentral/fileexchange/23611-peakfit-m).

Fitting Routine (785 nm and 532 nm data are fitted separately). Peak shapes, frequencies, and widths, for both wavelengths, in appendix A. An example command line code with detailed information is in appendix A for the peak fit parameters. Multilinear regression is utilized so fixed positions and widths can be applied to the peak fitting routine. A linear baseline correction is applied during the fit. 500 fits are performed during the fitting routine to minimize fitting error.

Data delivery. Data are compiled into an excel file with the position of Raman band as the columns and the samples as the rows.

### APPENDIX A—PEAK FITTING PARAMETERS

### Peakfit Command Line Example

[peakfit_results, Fit_stats]=peakfit([ramanshift spectral_Intensity’], center, window, NumPeaks, peakshape, extra, NumTrials, start, autozero, fitting_parameters_matrix);

### Output

“peakfit_results” is a matrix comprised of peak number, peak position (Raman shift frequencies), peak intensity, peak width (full width at half max), and peak area columns for each spectrum.

“Fit_stats” would be the goodness of fit statistics for the fit consisting of the root-mean-square error and R² value for each spectrum fitted.

### Input

“peakfit” is the command to execute the code of the peakfit.m (“Peakfit 9.0”) code in Matlab.

“ramanshift” is the x-value for the Raman spectrum being fit.

“spectral_intensity” is the intensity value at each pixel for the spectrum being fit.

“center” is a number value corresponding to the center frequency of the region being fit.

“window” is a number value corresponding to the width of the spectral window being fit.

“NumPeaks” corresponds to the number of peaks being fitted.

“peakshape” is a number value which corresponds to a specific peak shape in the peakfit code, ex. 1=Gaussian.

“extra” is a parameter which can fine-tune peak shapes. That is not used here.

“NumTrials” is a number value corresponding to the number of trials conducted by the computer in order to return the model that has the best fit.

“start” is a matrix of guess parameters used in a non-fixed parameter model. This is not used here.

“autozero” is a number value which corresponds to a specific type of baseline correction applied for the peak fitting model. These values are given in the peakfit code, ex. 1=linear.

“fitting_parameters_matrix” is the matrix of the fitting parameters. The current 785 nm and 532 nm models are given in Table 1 and Table 2, respectively. All peaks are assigned to be a Gaussian curve (peakshape=1).

### Peakfit Commands for 785 nm Data

Center=1891
Window=2511
NumPeaks=9
Peakshape=50

This corresponds to the multilinear regression peak shape which keeps all peak widths and positions fixed.
Extra=0
NumTrials=500
Start=0
Autozero=1

This corresponds to a linear baseline, which assumes that the signal returns to a baseline value of 0 at the edges of the window.

### Peakfit Commands for 532 nm Data

Center=1150
Window=1700
NumPeaks=12
Peakshape=50

This corresponds to the multilinear regression peak shape which keeps all peak widths and positions fixed.
Extra=0
NumTrials=500
Start=0
Autozero=0

Values of 0 correspond to that feature not being used in the model.

TABLE 1
Fitting parameters for 785 nm excitation spectra.

| Peak Shape | Frequency (cm⁻¹) | FWHM (Δcm⁻¹) |
|---|---|---|
| 1 | 969 | 42 |
| 1 | 1122 | 50 |
| 1 | 1199 | 100 |
| 1 | 1330 | 129 |
| 1 | 1400 | 76 |
| 1 | 1518 | 156 |
| 1 | 1605 | 130 |
| 1 | 1750 | 150 |
| 1 | 1825 | 1500 |

TABLE 2
Fitting parameters for 532 nm excitation spectra.

| Peak Shape | Frequency (cm⁻¹) | FWHM (Δcm⁻¹) |
|---|---|---|
| 1 | 462 | 165 |
| 1 | 592 | 148 |
| 1 | 763 | 174 |
| 1 | 856 | 27 |
| 1 | 985 | 50 |
| 1 | 1101 | 61 |
| 1 | 1213 | 122 |
| 1 | 1346 | 117 |
| 1 | 1416 | 69 |
| 1 | 1540 | 128 |
| 1 | 1613 | 81 |
| 1 | 1749 | 383 |

### Example 7—Protocol for Sample Handling, Protein Data Collection, and Processing

### Sample Handling and Preparation

Sample Storage. Samples are stored in −80 °C freezer before and after Raman analysis. Samples are removed from the freezer at least 30 minutes before Raman analysis to allow for thawing, and the un-aliquoted samples are replaced after analysis.

Sample Preparation. Once thawed, a small aliquot (approximately 3-10 strands) of hairs are transferred to a Platypus Technologies Aluminum coated microscope slide. The metal coating prevents glass interference and increases the Raman signal. Aluminum slides are rinsed with absolute ethanol and dried with a Kimwipe (lint free cleaning tissue) between hair samples.

### Instrumentation and Startup

Instrumentation. Data collection is performed on a Renishaw inVia Qontor Microscope (Renishaw plc). Raman excitation source is a 785 nm diode laser (Renishaw plc) with an output power of up to 300 mW (I 10% according to manufacturer). Laser light is focused on samples through a Leica N PLAN 50×L, 0.50 NA BD objective. Backscattered light is collected by the same objective, sent through a Rayleigh filter and diffracted by a 1200 lines/mm grating. Diffracted light is sent to a −70 °C (thermoelectrically cooled), 1024×256 pixel, Charge coupled device (CCD) detector.

Renishaw startup procedure (785 nm excitation). The laser is allowed to warm up for at least 30 minutes before alignment for pointing and stability.

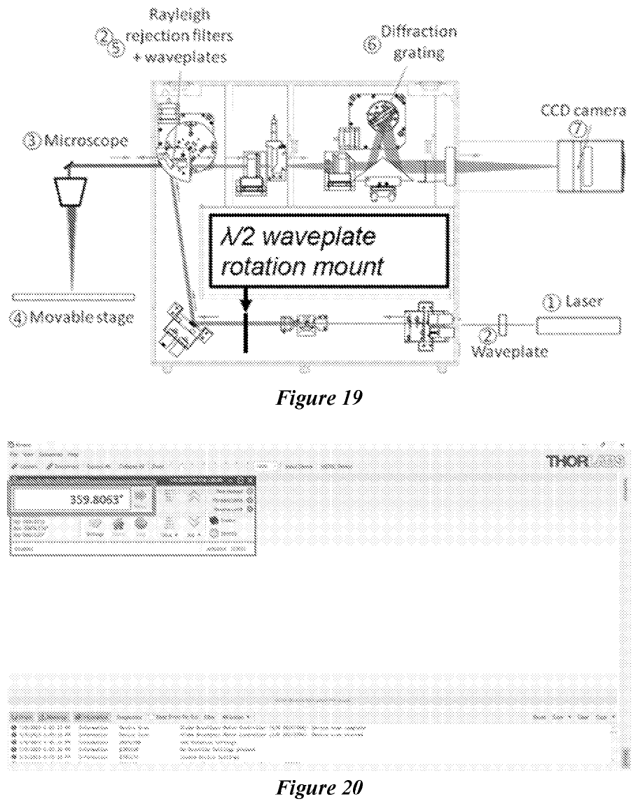

Laser polarization. A λ/2 waveplate has been mounted in the beam path (FIG. 19) on a ThorLabs DDR 25 rotation mount for polarization control in the x- and y-axes of the stage. The rotation mount is driven by a Thorlabs K-Cube brushless DC servo driver (KBD101) connected to Thorlabs Kinesis® software (FIG. 20). In the Kinesis interface, the KBD101 driver is enabled, homed, and the angles of the waveplate are controlled with the move feature.

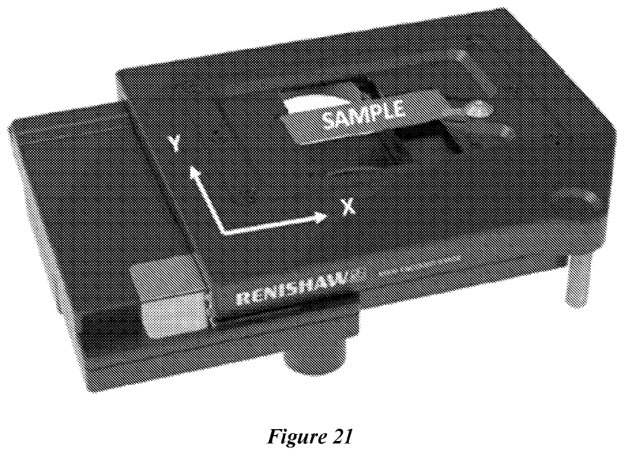

The natural polarization of the laser is in the x-direction of the stage (FIG. 21).

Currently, measurements are made with the laser polarization in the x- and y-directions with the sample axis in the x-direction. To determine the angle at which the waveplate must be to turn the beam into the y-direction, a polarizer is used in combination with the waveplate to determine the and angles at which the orthogonal polarization components are focused on the sample. The polarizer is aligned with the natural polarization of the laser, which generates an x-polarization at the sample. The polarizer alignment is verified using an incremented rotation stage, where the polarizer can be rotated 90 degrees to observe no transmission of the laser. The aligned polarizer is then moved after the rotating half-wave plate. The laser power is measured after the aligned polarizer as the waveplate is rotated. The angles that generate the maximum and minimum intensity are noted. If properly aligned, the maximum and minimum intensity will be 45 degrees different. The maximum intensity corresponds to the x-polarization and the minimum to y-polarization at the sample.

inVia auto-alignment procedures are performed in the following order as recommended by the instrument manufacturer: Silicon standard position; Laser alignment which optimizes the beamsteer motors; CCD confocal area which optimizes the area on the detector; The slit positions are optimized going into the spectrometer.

The calibration is corrected for drift by performing quick calibration which offsets the calibration, so the silicon phonon lands at 520.5 cm⁻¹.

785 nm laser performance Checks. Laser power is measured with a Thorlabs S130C slim power sensor. The power sensor is connected to a Thorlabs PM100A power meter console with mechanical and graphical displays. After alignment, the laser power at the sample is measured for the settings 100%, pinhole out and logged. On this system, this is around 100 mW at optimal performance. The laser power for data collection (100%, pinhole in) is also measured with the polarization in both the x- and y-directions at the sample. This can be used to correct detector counts for fluctuations in laser power. The typical laser power is 5 mW at the sample.

### Data Collection

Frequency calibration check standard. Acetaminophen is a published standard (ASTM E 1840) (Standard Guide for Raman Shift Standards Spectrometer Calibration. In Molecular Spectroscopy and Separation Science; Surface Analysis; Vol. ASTM 03.06). To check the calibration of the Renishaw Qontor, an acetaminophen sample is run at the start of each analysis day after startup. A high power (50% ND, pinhole out), 10 second exposure, extended scan (100-3200 cm⁻¹) is performed. In the WiRE software, each peak (in the fingerprint region 300-1800 cm⁻¹) is fitted and the frequencies are ensured to be within ±1 cm⁻¹ of their published values.

Hair Raman collection parameters. Scan type: Extended, over 300-1800 cm-1. Extended scans are being made over 300-3200 cm⁻¹ for discovery purposes. Exposure time: 120 seconds. Single acquisition per polarization. White light images are acquired with the inVia camera before and after spectral acquisition. Detector confocality: standard. Pinhole in. One scan collected with x-polarized light and one scan with y-polarized.

Sampling parameters. Samples are found in the microscope that have a root bulb (FIG. 16). To optimize polarization differences, hairs in the microscope are aligned in horizonal orientation as shown in FIG. 16. Focus is found on the sample in a spot that is clear of damage. For each mouse, three hairs data are collected as technical replicates.

### Data Processing

Software (WiRE™ 5) preprocessing. Cosmic rays are removed in WiRE™ 5 using the width of features algorithm. Cosmic ray-free files are saved as a separate .wdf file. Using the Batch File Converter (proprietary with WiRE™), cosmic ray-free files are converted to .txt for import into Matlab (Mathworks®).

Matlab preprocessing and analysis. Spectral data are imported into Matlab. The Raman shift values at each pixel are kept with each dataset for feature extraction. Data are baseline corrected using Rolling Circle Filter with a radius of 1,000,000 over the spectral region 400-1800 cm⁻¹ (Brandt et al. Optimization of the Rolling-Circle Filter for Raman Background Subtraction. Appl. Spectrosc., AS 2006, 60(3), 288-293). After baseline correction, data counts are standardized, considering laser polarization, by dividing by the measured laser power at the sample for that analysis day. After baseline correction, data are normalized to the δ(CH₂) mode ~1450 cm⁻¹ as a surrogate for the density of the hair sample.

### Feature Extraction and Data Delivery

Feature extraction. Curve fitting analysis with Peakfit 9.0 (Matlab program) is performed to extract intensity values from spectra in the 400-1800 cm⁻¹ region. A distribution of the program can be acquired from the MATLAB file exchange (https://www.mathworks.com/matlabcentral/fileexchange/23611-peakfit-m).

Fitting Routine (x and y-polarized spectra fitted separately). Peak shapes, frequencies, and widths, are listed in appendix B for x-polarization in Table 3 and y-polarization in Table 4 (note there appears to be an extra peak observed in y-polarization at 1479 cm⁻¹ Raman shift). An example command line code with detailed information is in appendix B for the peak fit parameters. Multilinear regression is utilized so fixed positions and widths can be applied to the peak fitting routine. No baseline correction is applied during the fit. 1000 fits are performed during the fitting routine to minimize fitting error.

Data delivery. Data are compiled into an excel file with the position of Raman band as the columns and the samples as the rows.

### APPENDIX B—PEAK FITTING PARAMETERS

### Peakfit Command Line Example

[peakfit_results,Fit_stats]=peakfit([ramanshift spectral_Intensity’],1100,1400,59,50,0,1000,0,0,fitting_parameters_matrix);

“peakfit_results” is a matrix comprised of peak number, peak position (Raman shift frequencies), peak intensity, peak width, and peak area columns for each spectrum.

“Fit_stats” would be the goodness of fit statistics for the fit consisting of the root-mean-square error and R² value for each spectrum fitted.

“peakfit” is the command to execute the code of the peakfit.m (“Peakfit 9.0”) code in Matlab.

“ramanshift” is the x-value for the Raman spectrum being fit.

“spectral_intensity” is the intensity value at each pixel for the spectrum being fit.

1100 is the centre frequency of the region being fit (400+1800/2).

1400 is the spectral window being fit (1800-400).

59 refers to the number of peaks in the fitting model.

50 refers to the multilinear regression fitting model which fixes the position and width for a defined peak shape.

The 0's refer to:

“extra” parameter which could fine tune peak shapes if the user chose. That is not used here.

“start” parameter which would be guess parameters used in a non-fixed parameter model.

“autozero” which refers to the type of baseline correction applied for the peak fitting model. Here it is 0 meaning no baseline since the spectral baseline was previously corrected (4.2.2).

1000 is the number of times the fit is applied and optimized.

“fitting_parameters_matrix” is the matrix of the fitting parameters. The current x- and y-polarized models are given in Table 3 and Table 4, respectively. In Table 3 and Table 4, peak shape 1 is Gaussian and 2 is Lorentzian.

TABLE 3
Fitting parameters for x-polarized excitation spectra.

| Peak Shape | Frequency (cm⁻¹) | FWHM (Δcm⁻¹) |
|---|---|---|
| 1 | 414 | 20 |
| 1 | 459 | 20 |
| 1 | 490 | 20 |
| 1 | 509 | 20 |
| 1 | 524 | 20 |
| 1 | 544 | 20 |
| 1 | 565 | 20 |
| 1 | 584 | 20 |
| 1 | 607 | 20 |
| 1 | 621.5 | 5 |
| 2 | 643.5 | 5 |
| 1 | 667 | 25 |
| 1 | 700 | 25 |
| 1 | 725 | 25 |
| 1 | 747 | 20 |
| 2 | 758 | 10 |
| 1 | 805 | 20 |
| 2 | 828.5 | 15 |
| 2 | 852 | 15 |
| 2 | 878 | 20 |
| 2 | 902 | 25 |
| 2 | 937 | 30 |
| 1 | 961 | 20 |
| 1 | 982 | 25 |
| 2 | 1004 | 7 |
| 2 | 1013 | 10 |
| 2 | 1023 | 12 |
| 2 | 1031 | 12 |
| 1 | 1044 | 20 |
| 1 | 1061 | 20 |
| 1 | 1081 | 20 |
| 1 | 1103 | 25 |
| 1 | 1128 | 15 |
| 1 | 1155 | 15 |
| 2 | 1175 | 15 |
| 1 | 1190 | 10 |
| 2 | 1208 | 20 |
| 1 | 1237 | 30 |
| 1 | 1267 | 35 |
| 1 | 1297 | 25 |
| 1 | 1317 | 35 |
| 1 | 1340 | 20 |
| 1 | 1360 | 20 |
| 1 | 1380 | 25 |
| 1 | 1405 | 25 |
| 1 | 1420 | 25 |
| 1 | 1440 | 20 |
| 1 | 1449 | 20 |
| 1 | 1465 | 25 |
| 1 | 1520 | 20 |
| 1 | 1553 | 15 |
| 1 | 1578 | 15 |
| 1 | 1597 | 15 |
| 2 | 1615 | 15 |
| 1 | 1634 | 20 |
| 1 | 1651 | 20 |
| 1 | 1671 | 30 |
| 1 | 1690 | 20 |
| 1 | 1725 | 20 |

TABLE 4
Fitting parameters for y-polarized excitation spectra.

| Peak Shape | Frequency (cm⁻¹) | FWHM (Δcm⁻¹) |
|---|---|---|
| 1 | 414 | 20 |
| 1 | 459 | 20 |
| 1 | 490 | 20 |
| 1 | 509 | 20 |
| 1 | 524 | 20 |
| 1 | 544 | 20 |
| 1 | 565 | 20 |
| 1 | 584 | 20 |
| 1 | 607 | 20 |
| 1 | 621.5 | 5 |
| 2 | 643.5 | 5 |
| 1 | 667 | 25 |
| 1 | 700 | 25 |
| 1 | 725 | 25 |
| 1 | 747 | 20 |
| 2 | 758 | 10 |
| 1 | 805 | 20 |
| 2 | 828.5 | 15 |
| 2 | 852 | 15 |
| 2 | 878 | 20 |
| 2 | 902 | 25 |
| 2 | 937 | 30 |
| 1 | 961 | 20 |
| 1 | 982 | 25 |
| 2 | 1004 | 7 |
| 2 | 1013 | 10 |
| 2 | 1023 | 12 |
| 2 | 1031 | 12 |
| 1 | 1044 | 20 |
| 1 | 1061 | 20 |
| 1 | 1081 | 20 |
| 1 | 1103 | 25 |
| 1 | 1128 | 15 |
| 1 | 1155 | 10 |
| 2 | 1175 | 15 |
| 1 | 1191 | 10 |
| 2 | 1208 | 20 |
| 1 | 1240 | 30 |
| 1 | 1269 | 35 |
| 1 | 1297 | 20 |
| 1 | 1315 | 30 |
| 1 | 1340 | 20 |
| 1 | 1360 | 20 |
| 1 | 1389 | 30 |
| 1 | 1420 | 30 |
| 1 | 1441 | 20 |
| 1 | 1450 | 20 |
| 1 | 1465 | 15 |
| 1 | 1479 | 20 |
| 1 | 1520 | 20 |
| 1 | 1553 | 15 |
| 1 | 1578 | 15 |
| 1 | 1597 | 15 |
| 2 | 1615 | 15 |
| 1 | 1634 | 20 |
| 1 | 1651 | 20 |
| 1 | 1671 | 30 |
| 1 | 1690 | 25 |
| 1 | 1725 | 20 |

Other advantages which are obvious and which are inherent to the invention will be evident to one skilled in the art. It will be understood that certain features and sub-combinations are of utility and may be employed without reference to other features and sub-combinations. This is contemplated by and is within the scope of the claims. Since many possible embodiments may be made of the invention without departing from the scope thereof, it is to be understood that all matter herein set forth or shown in the accompanying drawings is to be interpreted as illustrative and not in a limiting sense.

The methods of the appended claims are not limited in scope by the specific methods described herein, which are intended as illustrations of a few aspects of the claims and any methods that are functionally equivalent are intended to fall within the scope of the claims. Various modifications of the methods in addition to those shown and described herein are intended to fall within the scope of the appended claims. Further, while only certain representative method steps disclosed herein are specifically described, other combinations of the method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated.

1. A method comprising:
collecting a Raman signal from a sample; and
processing the Raman signal to determine a property of the sample;
wherein the sample comprises hair, skin, nails, or a combination thereof from a subject; and
wherein the property comprises the presence or absence of radiation exposure, the type of radiation, time since the radiation exposure, the duration of the exposure, the dose of the exposure, the dose-rate of exposure, or a combination thereof.

2. The method of claim 1, wherein the sample comprises hair, skin, or a combination thereof.

3. The method of claim 1, wherein the sample comprises hair.

4. The method of claim 1, wherein the sample comprises a hair root, a hair shaft, or a combination thereof.

5. The method of claim 1, wherein the type of radiation comprises ionizing radiation.

6. The method of claim 1, wherein the radiation exposure comprises exposure from neutrons, protons, electrons, photons, alpha-particles, beta-particles, charged particles, gamma rays, X-rays, chemotherapy, UV rays, or a combination thereof.

7. The method of claim 1, wherein the dose of the exposure is from 0.01 Gy to 20 Gy.

8. The method of claim 1, wherein the time since exposure is from 1 minute to 365 days.

9. The method of claim 1, wherein the dose-rate is from 0.1 to 1000 mGy/min.

10. The method of claim 1, wherein the dose-rate is from 0.1 to 1000 Gy/second.

11. The method of claim 1, wherein the amount of time from collecting the Raman signal to determining the property of the sample is from 1 second to 1 hour.

12. The method of claim 1, wherein at least a portion of the Raman signal corresponds to:
a vibrational frequency associated with radiation damage of the sample;
at least a portion of a protein that is susceptible to radiation damage;
a vibrational frequency associated with a disulfide group, a carbonyl group, or a combination thereof;
at least a portion of a pigment that is susceptible to radiation damage;
a vibrational frequency associated with a pigment;
or a combination thereof.

13. (canceled)

14. (canceled)

15. (canceled)

16. (canceled)

17. The method of claim 1, wherein at least a portion of the Raman signal provides evidence of aromatic amino acid modification.

18. The method of claim 1, wherein processing the Raman signal comprises determining the presence or absence of a signal, the intensity of a signal, the spectral shift of a signal, or a combination thereof.

19. (canceled)

20. (canceled)

21. (canceled)

22. The method of claim 1, wherein the method does not include collecting a baseline sample before exposure.

23. (canceled)

24. The method of claim 1, wherein the sample comprises hair and/or nails having a length, and the method further comprises collecting a plurality of Raman signals along the length of the sample, and processing the plurality of Raman signals to determine a property of the sample, wherein the length of the sample corresponds to a growth direction at growth rate, such that the property of the sample comprises time since the radiation exposure.

25. (canceled)

26. The method of claim 1, wherein the method is non-invasive.

27. (canceled)

28. (canceled)

29. The method of claim 1, wherein the method further comprises diagnosing and/or monitoring a condition in a subject based on the property of the sample.

30. The method of claim 29, further comprising selecting a course of therapy for the subject based on the property of the sample.

31. A device configured to perform the method of claim 1, the device comprising:
a receptacle configured to at least partially contain the sample;
an excitation source;
a detector; and
a computing device, wherein the computing device is configured to receive and process an electromagnetic signal from the detector;
wherein, when the device is assembled together with the sample, then:
the receptacle is configured to position the sample such that the sample is in optical communication with the excitation source and the detector;
the excitation source is configured to apply an excitation signal to the sample;
the detector is configured to collect the Raman signal from the sample; and
the computing device is configured to process the Raman signal to determine the property of the sample.

32.-38. (canceled)
